SEQUENCE LISTING (226 sequences)

SEQ ID NO: 1 - DNA, 17 bp, Artificial Sequence
  Description of Artificial Sequence Synthetic primer
ctgggcgagaacaacga

SEQ ID NO: 2 - DNA, 26 bp, Artificial Sequence
  Description of Artificial Sequence Synthetic probe
actcctcgaagtacacgtagcccccg

SEQ ID NO: 3 - DNA, 21 bp, Artificial Sequence
  Description of Artificial Sequence Synthetic primer
gatgttcaggtcgatgaaggt

SEQ ID NO: 4 - DNA, 17 bp, Artificial Sequence
  Description of Artificial Sequence Synthetic primer
gccacggtggtgcagtt

SEQ ID NO: 5 - DNA, 22 bp, Artificial Sequence
  Description of Artificial Sequence Synthetic probe
ccttgaagaccaccgcgatgcc

SEQ ID NO: 6 - DNA, 18 bp, Artificial Sequence
  Description of Artificial Sequence Synthetic primer
tgtacggggcgatgttct

SEQ ID NO: 7 - DNA, 21 bp, Artificial Sequence
  Description of Artificial Sequence Synthetic probe
aagaccaccgcgatgccctcc

SEQ ID NO: 8 - DNA, 26 bp, Artificial Sequence
  Description of Artificial Sequence Synthetic probe
accgcgatgccctccgtgtagttctg

SEQ ID NO: 9 - DNA, 23 bp, Artificial Sequence
  Description of Artificial Sequence Synthetic probe
tccttgaagaccaccgcgatgcc

SEQ ID NO: 10 - DNA, 18 bp, Artificial Sequence
  Description of Artificial Sequence Synthetic primer
ttgtacggggcgatgttc

SEQ ID NO: 11 - DNA, 22 bp, Artificial Sequence
  Description of Artificial Sequence Synthetic probe
aagaccaccgcgatgccctccg

SEQ ID NO: 12 - DNA, 24 bp, Artificial Sequence
  Description of Artificial Sequence Synthetic primer
tttgtagtacatggtggccttgaa

SEQ ID NO: 13 - DNA, 19 bp, Artificial Sequence
  Description of Artificial Sequence Synthetic primer
ttgtacggggcgatgttct

SEQ ID NO: 14 - DNA, 24 bp, Artificial Sequence
  Description of Artificial Sequence Synthetic primer
ctttgtagtacatggtggccttga

SEQ ID NO: 15 - DNA, 24 bp, Artificial Sequence
  Description of Artificial Sequence Synthetic probe
tcttcaaggagaacatcgccccgt

SEQ ID NO: 16 - DNA, 17 bp, Artificial Sequence
  Description of Artificial Sequence Synthetic primer
gcatcgcggtggtcttc

SEQ ID NO: 17 - DNA, 25 bp, Artificial Sequence
  Description of Artificial Sequence Synthetic probe
caggtgtggttcggccaccgctact

SEQ ID NO: 18 - DNA, 19 bp, Artificial Sequence
  Description of Artificial Sequence Synthetic primer
tgtcgatcacctcctcgaa

SEQ ID NO: 19 - DNA, 21 bp, Artificial Sequence
  Description of Artificial Sequence Synthetic primer
aaaacggggacatgtacacaa

SEQ ID NO: 20 - DNA, 23 bp, Artificial Sequence
  Description of Artificial Sequence Synthetic primer
tcaaggccaccatgtactacaaa

SEQ ID NO: 21 - DNA, 24 bp, Artificial Sequence
  Description of Artificial Sequence Synthetic probe
caggtgtggttcggccaccgctac

SEQ ID NO: 22 - DNA, 23 bp, Artificial Sequence
  Description of Artificial Sequence Synthetic primer
gtaaaacggggacatgtacacaa

SEQ ID NO: 23 - DNA, 25 bp, Artificial Sequence
  Description of Artificial Sequence Synthetic probe
aggtgtggttcggccaccgctactc

SEQ ID NO: 24 - DNA, 24 bp, Artificial Sequence
  Description of Artificial Sequence Synthetic primer
caaggccaccatgtactacaaaga

SEQ ID NO: 25 - DNA, 18 bp, Artificial Sequence
  Description of Artificial Sequence Synthetic primer
gaacatcgccccgtacaa

SEQ ID NO: 26 - DNA, 23 bp, Artificial Sequence
  Description of Artificial Sequence Synthetic probe
tgtggttcggccaccgctactcc

SEQ ID NO: 27 - DNA, 23 bp, Artificial Sequence
  Description of Artificial Sequence Synthetic probe
tggttcggccaccgctactccca

SEQ ID NO: 28 - DNA, 27 bp, Artificial Sequence
  Description of Artificial Sequence Synthetic probe
ttcggccaccgctactcccagtttatg

SEQ ID NO: 29 - DNA, 26 bp, Artificial Sequence
  Description of Artificial Sequence Synthetic probe
cggccaccgctactcccagtttatgg

SEQ ID NO: 30 - DNA, 18 bp, Artificial Sequence
  Description of Artificial Sequence Synthetic primer
cccgcgaggggttgtact

SEQ ID NO: 31 - DNA, 28 bp, Artificial Sequence
  Description of Artificial Sequence Synthetic probe
tggtcttcaaggagaacatcgccccgta

SEQ ID NO: 32 - DNA, 28 bp, Artificial Sequence
  Description of Artificial Sequence Synthetic probe
tcttcaaggagaacatcgccccgtacaa

SEQ ID NO: 33 - DNA, 19 bp, Artificial Sequence
  Description of Artificial Sequence Synthetic primer
tcgaggaggtgatcgacaa

SEQ ID NO: 34 - DNA, 22 bp, Artificial Sequence
  Description of Artificial Sequence Synthetic probe
tggagaccaccgcgtttcaccg

SEQ ID NO: 35 - DNA, 21 bp, Artificial Sequence
  Description of Artificial Sequence Synthetic primer
acggggacatgtacacaaagt

SEQ ID NO: 36 - DNA, 25 bp, Artificial Sequence
  Description of Artificial Sequence Synthetic primer
actttgtgtacatgtccccgtttta

SEQ ID NO: 37 - DNA, 20 bp, Artificial Sequence
  Description of Artificial Sequence Synthetic probe
tacgccgccgaccgcttcaa

SEQ ID NO: 38 - DNA, 19 bp, Artificial Sequence
  Description of Artificial Sequence Synthetic primer
ggaaggagccgccgtactc

SEQ ID NO: 39 - DNA, 18 bp, Artificial Sequence
  Description of Artificial Sequence Synthetic primer
gaaggagccgccgtactc

SEQ ID NO: 40 - DNA, 20 bp, Artificial Sequence
  Description of Artificial Sequence Synthetic primer
tcctgccacttggtcatggt

SEQ ID NO: 41 - DNA, 18 bp, Artificial Sequence
  Description of Artificial Sequence Synthetic primer
cgcgaggggttgtacttg

SEQ ID NO: 42 - DNA, 24 bp, Artificial Sequence
  Description of Artificial Sequence Synthetic primer
tttgtgtacatgtccccgttttac

SEQ ID NO: 43 - DNA, 20 bp, Artificial Sequence
  Description of Artificial Sequence Synthetic primer
cgaggaggtgatcgacaaga

SEQ ID NO: 44 - DNA, 17 bp, Artificial Sequence
  Description of Artificial Sequence Synthetic primer
ccgcgaggggttgtact

SEQ ID NO: 45 - DNA, 17 bp, Artificial Sequence
  Description of Artificial Sequence Synthetic primer
ggagaccaccgcgtttc

SEQ ID NO: 46 - DNA, 28 bp, Artificial Sequence
  Description of Artificial Sequence Synthetic probe
caccaccgacctcaagtacaacccctcg

SEQ ID NO: 47 - DNA, 19 bp, Artificial Sequence
  Description of Artificial Sequence Synthetic primer
agccgtaaaacggggacat

SEQ ID NO: 48 - DNA, 22 bp, Artificial Sequence
  Description of Artificial Sequence Synthetic primer
taaaacggggacatgtacacaa

SEQ ID NO: 49 - DNA, 19 bp, Artificial Sequence
  Description of Artificial Sequence Synthetic primer
acccgcgaggggttgtact

SEQ ID NO: 50 - DNA, 25 bp, Artificial Sequence
  Description of Artificial Sequence Synthetic primer
tcaaggccaccatgtactacaaaga

SEQ ID NO: 51 - DNA, 22 bp, Artificial Sequence
  Description of Artificial Sequence Synthetic primer
caaggccaccatgtactacaaa

SEQ ID NO: 52 - DNA, 21 bp, Artificial Sequence
  Description of Artificial Sequence Synthetic primer
cttgtcgatcacctcctcgaa

SEQ ID NO: 53 - DNA, 22 bp, Artificial Sequence
  Description of Artificial Sequence Synthetic primer
tcttgtcgatcacctcctcgaa

SEQ ID NO: 54 - DNA, 18 bp, Artificial Sequence
  Description of Artificial Sequence Synthetic primer
agcggcctgctggactac

SEQ ID NO: 55 - DNA, 20 bp, Artificial Sequence
  Description of Artificial Sequence Synthetic probe
aggtccagcgccgcaaccag

SEQ ID NO: 56 - DNA, 19 bp, Artificial Sequence
  Description of Artificial Sequence Synthetic primer
ggatgaccgtgtcgatgtc

SEQ ID NO: 57 - DNA, 22 bp, Artificial Sequence
  Description of Artificial Sequence Synthetic primer
aacggggacatgtacacaaagt

SEQ ID NO: 58 - DNA, 23 bp, Artificial Sequence
  Description of Artificial Sequence Synthetic primer
ttcgaggaggtgatcgacaagat

SEQ ID NO: 59 - DNA, 17 bp, Artificial Sequence
  Description of Artificial Sequence Synthetic primer
tggagaccaccgcgttt

SEQ ID NO: 60 - DNA, 26 bp, Artificial Sequence
  Description of Artificial Sequence Synthetic probe
caccgacctcaagtacaacccctcgc

SEQ ID NO: 61 - DNA, 20 bp, Artificial Sequence
  Description of Artificial Sequence Synthetic primer
acccgcgaggggttgtactt

SEQ ID NO: 62 - DNA, 18 bp, Artificial Sequence
  Description of Artificial Sequence Synthetic primer
cctcccggtagccgtaaa

SEQ ID NO: 63 - DNA, 21 bp, Artificial Sequence
  Description of Artificial Sequence Synthetic probe
ccgcaaccagctgcacgacct

SEQ ID NO: 64 - DNA, 17 bp, Artificial Sequence
  Description of Artificial Sequence Synthetic primer
agcggcctgctggacta

SEQ ID NO: 65 - DNA, 25 bp, Artificial Sequence
  Description of Artificial Sequence Synthetic primer
caaggccaccatgtactacaaagac

SEQ ID NO: 66 - DNA, 26 bp, Artificial Sequence
  Description of Artificial Sequence Synthetic primer
actttgtgtacatgtccccgttttac

SEQ ID NO: 67 - DNA, 24 bp, Artificial Sequence
  Description of Artificial Sequence Synthetic probe
cttctacgcgcgcgacctcaccac

SEQ ID NO: 68 - DNA, 24 bp, Artificial Sequence
  Description of Artificial Sequence Synthetic primer
gtaaaacggggacatgtacacaaa

SEQ ID NO: 69 - DNA, 23 bp, Artificial Sequence
  Description of Artificial Sequence Synthetic probe
ccgcgatgccctccgtgtagttc

SEQ ID NO: 70 - DNA, 22 bp, Artificial Sequence
  Description of Artificial Sequence Synthetic primer
cgaggaggaggggtataacaaa

SEQ ID NO: 71 - DNA, 25 bp, Artificial Sequence
  Description of Artificial Sequence Synthetic probe
aagcttcagcgcgaacgaccaacta

SEQ ID NO: 72 - DNA, 17 bp, Artificial Sequence
  Description of Artificial Sequence Synthetic primer
atcacggcccccaacct

SEQ ID NO: 73 - DNA, 18 bp, Artificial Sequence
  Description of Artificial Sequence Synthetic primer
agacgcccctccctgtgt

SEQ ID NO: 74 - DNA, 22 bp, Artificial Sequence
  Description of Artificial Sequence Synthetic probe
tcagtcgacccaagcgcggaac

SEQ ID NO: 75 - DNA, 20 bp, Artificial Sequence
  Description of Artificial Sequence Synthetic primer
tctcgtcggggggagtagtg

SEQ ID NO: 76 - DNA, 18 bp, Artificial Sequence
  Description of Artificial Sequence Synthetic primer
tccgaagacgtccggaaa

SEQ ID NO: 77 - DNA, 24 bp, Artificial Sequence
  Description of Artificial Sequence Synthetic probe
cctcccatccgaaaccaagcgatg

SEQ ID NO: 78 - DNA, 21 bp, Artificial Sequence
  Description of Artificial Sequence Synthetic probe
cgtgatggggatagcacagtt

SEQ ID NO: 79 - DNA, 18 bp, Artificial Sequence
  Description of Artificial Sequence Synthetic primer
cgtcagcccatcctcctt

SEQ ID NO: 80 - DNA, 20 bp, Artificial Sequence
  Description of Artificial Sequence Synthetic probe
ccgtccccaaagacgtgcgg

SEQ ID NO: 81 - DNA, 20 bp, Artificial Sequence
  Description of Artificial Sequence Synthetic primer
cagcagggaagcatttacga

SEQ ID NO: 82 - DNA, 21 bp, Artificial Sequence
  Description of Artificial Sequence Synthetic primer
ccggaaacaaccctacaacct

SEQ ID NO: 83 - DNA, 20 bp, Artificial Sequence
  Description of Artificial Sequence Synthetic primer
gtgccgttgttcccattatc

SEQ ID NO: 84 - DNA, 30 bp, Artificial Sequence
  Description of Artificial Sequence Synthetic probe
ccattccttttggttcttgtcggtgtatcg

SEQ ID NO: 85 - DNA, 19 bp, Artificial Sequence
  Description of Artificial Sequence Synthetic primer
gggtggtggaggagacgtt

SEQ ID NO: 86 - DNA, 28 bp, Artificial Sequence
  Description of Artificial Sequence Synthetic probe
cgacccggtacgctctcgtaaatgcttc

SEQ ID NO: 87 - DNA, 19 bp, Artificial Sequence
  Description of Artificial Sequence Synthetic primer
cgccgagttcgatctggta

SEQ ID NO: 88 - DNA, 18 bp, Artificial Sequence
  Description of Artificial Sequence Synthetic primer
ccacgagaccgacatgga

SEQ ID NO: 89 - DNA, 19 bp, Artificial Sequence
  Description of Artificial Sequence Synthetic primer
ctcgtcggggggagtagtg

SEQ ID NO: 90 - DNA, 10 bp, Artificial Sequence
  Description of Artificial Sequence Synthetic probe
aaacacgtgc

SEQ ID NO: 91 - DNA, 10 bp, Artificial Sequence
  Description of Artificial Sequence Synthetic probe
ccttgttcca

SEQ ID NO: 92 - DNA, 10 bp, Artificial Sequence
  Description of Artificial Sequence Synthetic probe
cagggacgat

SEQ ID NO: 93 - DNA, 25 bp, Artificial Sequence
  Description of Artificial Sequence Synthetic primer
cattttacgaggaggaggggtataa

SEQ ID NO: 94 - DNA, 24 bp, Artificial Sequence
  Description of Artificial Sequence Synthetic probe
aagcttcagcgcgaacgaccaact

SEQ ID NO: 95 - DNA, 18 bp, Artificial Sequence
  Description of Artificial Sequence Synthetic primer
aatcacggcccccaacct

SEQ ID NO: 96 - DNA, 19 bp, Artificial Sequence
  Description of Artificial Sequence Synthetic primer
aagacgcccctccctgtgt

SEQ ID NO: 97 - DNA, 21 bp, Artificial Sequence
  Description of Artificial Sequence Synthetic probe
tcagtcgacccaagcgcggaa

SEQ ID NO: 98 - DNA, 20 bp, Artificial Sequence
  Description of Artificial Sequence Synthetic primer
catctcgtcggggggagtag

SEQ ID NO: 99 - DNA, 23 bp, Artificial Sequence
  Description of Artificial Sequence Synthetic primer
tacgaggaggaggggtataacaa

SEQ ID NO: 100 - DNA, 19 bp, Artificial Sequence
  Description of Artificial Sequence Synthetic primer
tactccccccgacgagatg

SEQ ID NO: 101 - DNA, 21 bp, Artificial Sequence
  Description of Artificial Sequence Synthetic probe
ccacacaagccgcaacggtcg

SEQ ID NO: 102 - DNA, 17 bp, Artificial Sequence
  Description of Artificial Sequence Synthetic primer
ggaggcgactgccgttt

SEQ ID NO: 103 - DNA, 16 bp, Artificial Sequence
  Description of Artificial Sequence Synthetic primer
gggggaggggccattt

SEQ ID NO: 104 - DNA, 25 bp, Artificial Sequence
  Description of Artificial Sequence Synthetic probe
cggtcataagcttcagcgcgaacga

SEQ ID NO: 105 - DNA, 18 bp, Artificial Sequence
  Description of Artificial Sequence Synthetic primer
actccccccgacgagatg

SEQ ID NO: 106 - DNA, 26 bp, Artificial Sequence
  Description of Artificial Sequence Synthetic probe
tcataagcttcagcgcgaacgaccaa

SEQ ID NO: 107 - DNA, 20 bp, Artificial Sequence
  Description of Artificial Sequence Synthetic primer
actactccccccgacgagat

SEQ ID NO: 108 - DNA, 18 bp, Artificial Sequence
  Description of Artificial Sequence Synthetic primer
gggaggggccattttacg

SEQ ID NO: 109 - DNA, 28 bp, Artificial Sequence
  Description of Artificial Sequence Synthetic probe
ataagcttcagcgcgaacgaccaactac

SEQ ID NO: 110 - DNA, 16 bp, Artificial Sequence
  Description of Artificial Sequence Synthetic primer
tcacggcccccaacct

SEQ ID NO: 111 - DNA, 18 bp, Artificial Sequence
  Description of Artificial Sequence Synthetic primer
cccccgcaaccactactc

SEQ ID NO: 112 - DNA, 28 bp, Artificial Sequence
  Description of Artificial Sequence Synthetic probe
cataagcttcagcgcgaacgaccaacta

SEQ ID NO: 113 - DNA, 19 bp, Artificial Sequence
  Description of Artificial Sequence Synthetic primer
ctactccccccgacgagat

SEQ ID NO: 114 - DNA, 26 bp, Artificial Sequence
  Description of Artificial Sequence Synthetic probe
aagcttcagcgcgaacgaccaactac

SEQ ID NO: 115 - DNA, 29 bp, Artificial Sequence
  Description of Artificial Sequence Synthetic probe
tcataagcttcagcgcgaacgaccaacta

SEQ ID NO: 116 - DNA, 18 bp, Artificial Sequence
  Description of Artificial Sequence Synthetic primer
ggggaggggccattttac

SEQ ID NO: 117 - DNA, 17 bp, Artificial Sequence
  Description of Artificial Sequence Synthetic primer
actccccccgacgagat

SEQ ID NO: 118 - DNA, 23 bp, Artificial Sequence
  Description of Artificial Sequence Synthetic primer
acgaggaggaggggtataacaaa

SEQ ID NO: 119 - DNA, 23 bp, Artificial Sequence
  Description of Artificial Sequence Synthetic probe
aagcttcagcgcgaacgaccaac

SEQ ID NO: 120 - DNA, 19 bp, Artificial Sequence
  Description of Artificial Sequence Synthetic primer
catctcgtcggggggagta

SEQ ID NO: 121 - DNA, 26 bp, Artificial Sequence
  Description of Artificial Sequence Synthetic probe
cataagcttcagcgcgaacgaccaac

SEQ ID NO: 122 - DNA, 27 bp, Artificial Sequence
  Description of Artificial Sequence Synthetic probe
cataagcttcagcgcgaacgaccaact

SEQ ID NO: 123 - DNA, 23 bp, Artificial Sequence
  Description of Artificial Sequence Synthetic probe
agcttcagcgcgaacgaccaact

SEQ ID NO: 124 - DNA, 20 bp, Artificial Sequence
  Description of Artificial Sequence Synthetic probe
cagtcgacccaagcgcggaa

SEQ ID NO: 125 - DNA, 18 bp, Artificial Sequence
  Description of Artificial Sequence Synthetic primer
tcgtcggggggagtagtg

SEQ ID NO: 126 - DNA, 21 bp, Artificial Sequence
  Description of Artificial Sequence Synthetic probe
ctcagtcgacccaagcgcgga

SEQ ID NO: 127 - DNA, 20 bp, Artificial Sequence
  Description of Artificial Sequence Synthetic primer
atctcgtcggggggagtagt

SEQ ID NO: 128 - DNA, 23 bp, Artificial Sequence
  Description of Artificial Sequence Synthetic primer
cgaggaggaggggtataacaaag

SEQ ID NO: 129 - DNA, 21 bp, Artificial Sequence
  Description of Artificial Sequence Synthetic probe
cagtcgacccaagcgcggaac

SEQ ID NO: 130 - DNA, 19 bp, Artificial Sequence
  Description of Artificial Sequence Synthetic primer
tctcgtcggggggagtagt

SEQ ID NO: 131 - DNA, 18 bp, Artificial Sequence
  Description of Artificial Sequence Synthetic primer
acctgcggctcgtgaaga

SEQ ID NO: 132 - DNA, 32 bp, Artificial Sequence
  Description of Artificial Sequence Synthetic probe
aaacgactggacggagattacacagtttatcc

SEQ ID NO: 133 - DNA, 18 bp, Artificial Sequence
  Description of Artificial Sequence Synthetic primer
cgtcaccccctgctggta

SEQ ID NO: 134 - DNA, 23 bp, Artificial Sequence
  Description of Artificial Sequence Synthetic primer
aaacgactggacggagattacac

SEQ ID NO: 135 - DNA, 26 bp, Artificial Sequence
  Description of Artificial Sequence Synthetic probe
agtttatcctggagcaccgagccaag

SEQ ID NO: 136 - DNA, 24 bp, Artificial Sequence
  Description of Artificial Sequence Synthetic primer
aagataaacgactggacggagatt

SEQ ID NO: 137 - DNA, 27 bp, Artificial Sequence
  Description of Artificial Sequence Synthetic probe
acacagtttatcctggagcaccgagcc

SEQ ID NO: 138 - DNA, 25 bp, Artificial Sequence
  Description of Artificial Sequence Synthetic primer
ataaacgactggacggagattacac

SEQ ID NO: 139 - DNA, 20 bp, Artificial Sequence
  Description of Artificial Sequence Synthetic primer
gccccgctggaactactatg

SEQ ID NO: 140 - DNA, 22 bp, Artificial Sequence
  Description of Artificial Sequence Synthetic probe
acagcttcagcgccgtcagcga

SEQ ID NO: 141 - DNA, 16 bp, Artificial Sequence
  Description of Artificial Sequence Synthetic primer
gtgccggcggtctcaa

SEQ ID NO: 142 - DNA, 24 bp, Artificial Sequence
  Description of Artificial Sequence Synthetic primer
gtgtaatctccgtccagtcgttta

SEQ ID NO: 143 - DNA, 16 bp, Artificial Sequence
  Description of Artificial Sequence Synthetic primer
ccggcggtctcaaacg

SEQ ID NO: 144 - DNA, 16 bp, Artificial Sequence
  Description of Artificial Sequence Synthetic primer
gccgtcagcccatcct

SEQ ID NO: 145 - DNA, 20 bp, Artificial Sequence
  Description of Artificial Sequence Synthetic primer
cgaagacgtccggaaacaac

SEQ ID NO: 146 - DNA, 26 bp, Artificial Sequence
  Description of Artificial Sequence Synthetic probe
acagttgcctcccatccgaaaccaag

SEQ ID NO: 147 - DNA, 20 bp, Artificial Sequence
  Description of Artificial Sequence Synthetic primer
atgaccgtgatggggatagc

SEQ ID NO: 148 - DNA, 19 bp, Artificial Sequence
  Description of Artificial Sequence Synthetic primer
cgaagacgtccggaaacaa

SEQ ID NO: 149 - DNA, 18 bp, Artificial Sequence
  Description of Artificial Sequence Synthetic primer
gaggccccccagattgtc

SEQ ID NO: 150 - DNA, 19 bp, Artificial Sequence
  Description of Artificial Sequence Synthetic primer
cctcccgatcacggtttac

SEQ ID NO: 151 - DNA, 20 bp, Artificial Sequence
  Description of Artificial Sequence Synthetic probe
cctgccgcagcgtgctccta

SEQ ID NO: 152 - DNA, 16 bp, Artificial Sequence
  Description of Artificial Sequence Synthetic primer
ccccgcggacaatctg

SEQ ID NO: 153 - DNA, 21 bp, Artificial Sequence
  Description of Artificial Sequence Synthetic primer
agccccgctggaactactatg

SEQ ID NO: 154 - DNA, 22 bp, Artificial Sequence
  Description of Artificial Sequence Synthetic probe
cagcttcagcgccgtcagcgag

SEQ ID NO: 155 - DNA, 20 bp, Artificial Sequence
  Description of Artificial Sequence Synthetic primer
atcttcacgagccgcaggta

SEQ ID NO: 156 - DNA, 18 bp, Artificial Sequence
  Description of Artificial Sequence Synthetic primer
acggcctcccctgctcta

SEQ ID NO: 157 - DNA, 34 bp, Artificial Sequence
  Description of Artificial Sequence Synthetic probe
atatcctctttatcatcagcaccaccatccacac

SEQ ID NO: 158 - DNA, 20 bp, Artificial Sequence
  Description of Artificial Sequence Synthetic primer
aaggcgaccagacaaacgaa

SEQ ID NO: 159 - DNA, 25 bp, Artificial Sequence
  Description of Artificial Sequence Synthetic primer
ctcccctgctctagatatcctcttt

SEQ ID NO: 160 - DNA, 25 bp, Artificial Sequence
  Description of Artificial Sequence Synthetic probe
atcatcagcaccaccatccacacgg

SEQ ID NO: 161 - DNA, 27 bp, Artificial Sequence
  Description of Artificial Sequence Synthetic primer
ctcccctgctctagatatcctctttat

SEQ ID NO: 162 - DNA, 24 bp, Artificial Sequence
  Description of Artificial Sequence Synthetic probe
catcagcaccaccatccacacggc

SEQ ID NO: 163 - DNA, 27 bp, Artificial Sequence
  Description of Artificial Sequence Synthetic primer
tcccctgctctagatatcctctttatc

SEQ ID NO: 164 - DNA, 23 bp, Artificial Sequence
  Description of Artificial Sequence Synthetic probe
atcagcaccaccatccacacggc

SEQ ID NO: 165 - DNA, 28 bp, Artificial Sequence
  Description of Artificial Sequence Synthetic primer
ccctgctctagatatcctctttatcatc

SEQ ID NO: 166 - DNA, 22 bp, Artificial Sequence
  Description of Artificial Sequence Synthetic probe
agcaccaccatccacacggcgg

SEQ ID NO: 167 - DNA, 19 bp, Artificial Sequence
  Description of Artificial Sequence Synthetic primer
caaggcgaccagacaaacg

SEQ ID NO: 168 - DNA, 20 bp, Artificial Sequence
  Description of Artificial Sequence Synthetic primer
cacatccccctgttctggtt

SEQ ID NO: 169 - DNA, 31 bp, Artificial Sequence
  Description of Artificial Sequence Synthetic probe
cctaacggcctcccctgctctagatatcctc

SEQ ID NO: 170 - DNA, 22 bp, Artificial Sequence
  Description of Artificial Sequence Synthetic primer
ggatggtggtgctgatgataaa

SEQ ID NO: 171 - DNA, 19 bp, Artificial Sequence
  Description of Artificial Sequence Synthetic primer
cccaacacatccccctgtt

SEQ ID NO: 172 - DNA, 28 bp, Artificial Sequence
  Description of Artificial Sequence Synthetic probe
ctggttcctaacggcctcccctgctcta

SEQ ID NO: 173 - DNA, 21 bp, Artificial Sequence
  Description of Artificial Sequence Synthetic primer
acacatccccctgttctggtt

SEQ ID NO: 174 - DNA, 32 bp, Artificial Sequence
  Description of Artificial Sequence Synthetic probe
cctaacggcctcccctgctctagatatcctct

SEQ ID NO: 175 - DNA, 22 bp, Artificial Sequence
  Description of Artificial Sequence Synthetic primer
tggatggtggtgctgatgataa

SEQ ID NO: 176 - DNA, 17 bp, Artificial Sequence
  Description of Artificial Sequence Synthetic primer
ggcggcgttcgtttgtc

SEQ ID NO: 177 - DNA, 24 bp, Artificial Sequence
  Description of Artificial Sequence Synthetic probe
tggtcgccttggcagcacaacttt

SEQ ID NO: 178 - DNA, 16 bp, Artificial Sequence
  Description of Artificial Sequence Synthetic primer
tcgggtgcgcgtatcg

SEQ ID NO: 179 - DNA, 23 bp, Artificial Sequence
  Description of Artificial Sequence Synthetic probe
acagcttcagcgccgtcagcgag

SEQ ID NO: 180 - DNA, 25 bp, Artificial Sequence
  Description of Artificial Sequence Synthetic primer
ggtggtgctgatgataaagaggata

SEQ ID NO: 181 - DNA, 19 bp, Artificial Sequence
  Description of Artificial Sequence Synthetic primer
tcttcacgagccgcaggta

SEQ ID NO: 182 - DNA, 21 bp, Artificial Sequence
  Description of Artificial Sequence Synthetic primer
tatcttcacgagccgcaggta

SEQ ID NO: 183 - DNA, 36 bp, Artificial Sequence
  Description of Artificial Sequence Synthetic probe
cctaacggcctcccctgctctagatatcctctttat

SEQ ID NO: 184 - DNA, 16 bp, Artificial Sequence
  Description of Artificial Sequence Synthetic primer
ccgccgtgtggatggt

SEQ ID NO: 185 - DNA, 31 bp, Artificial Sequence
  Description of Artificial Sequence Synthetic probe
ctaacggcctcccctgctctagatatcctct

SEQ ID NO: 186 - DNA, 20 bp, Artificial Sequence
  Description of Artificial Sequence Synthetic primer
gtggatggtggtgctgatga

SEQ ID NO: 187 - DNA, 32 bp, Artificial Sequence
  Description of Artificial Sequence Synthetic probe
atatcctctttatcatcagcaccaccatccac

SEQ ID NO: 188 - DNA, 23 bp, Artificial Sequence
  Description of Artificial Sequence Synthetic probe
catcagcaccaccatccacacgg

SEQ ID NO: 189 - DNA, 18 bp, Artificial Sequence
  Description of Artificial Sequence Synthetic primer
cctgcggctcgtgaagat

SEQ ID NO: 190 - DNA, 18 bp, Artificial Sequence
  Description of Artificial Sequence Synthetic primer
ccccacaccccaacacat

SEQ ID NO: 191 - DNA, 24 bp, Artificial Sequence
  Description of Artificial Sequence Synthetic probe
ccccctgttctggttcctaacggc

SEQ ID NO: 192 - DNA, 16 bp, Artificial Sequence
  Description of Artificial Sequence Synthetic primer
gccccacaccccaaca

SEQ ID NO: 193 - DNA, 26 bp, Artificial Sequence
  Description of Artificial Sequence Synthetic probe
catccccctgttctggttcctaacgg

SEQ ID NO: 194 - DNA, 24 bp, Artificial Sequence
  Description of Artificial Sequence Synthetic primer
gaagataaacgactggacggagat

SEQ ID NO: 195 - DNA, 30 bp, Artificial Sequence
  Description of Artificial Sequence Synthetic probe
acacagtttatcctggagcaccgagccaag

SEQ ID NO: 196 - DNA, 33 bp, Artificial Sequence
  Description of Artificial Sequence Synthetic probe
atatcctctttatcatcagcaccaccatccaca

SEQ ID NO: 197 - DNA, 20 bp, Artificial Sequence
  Description of Artificial Sequence Synthetic primer
tgtggatggtggtgctgatg

SEQ ID NO: 198 - DNA, 30 bp, Artificial Sequence
  Description of Artificial Sequence Synthetic probe
tacacagtttatcctggagcaccgagccaa

SEQ ID NO: 199 - DNA, 24 bp, Artificial Sequence
  Description of Artificial Sequence Synthetic primer
taaacgactggacggagattacac

SEQ ID NO: 200 - DNA, 25 bp, Artificial Sequence
  Description of Artificial Sequence Synthetic primer
gataaacgactggacggagattaca

SEQ ID NO: 201 - DNA, 27 bp, Artificial Sequence
  Description of Artificial Sequence Synthetic probe
cagtttatcctggagcaccgagccaag

SEQ ID NO: 202 - DNA, 26 bp, Artificial Sequence
  Description of Artificial Sequence Synthetic probe
ccccctgttctggttcctaacggcct

SEQ ID NO: 203 - DNA, 25 bp, Artificial Sequence
  Description of Artificial Sequence Synthetic primer
aagataaacgactggacggagatta

SEQ ID NO: 204 - DNA, 29 bp, Artificial Sequence
  Description of Artificial Sequence Synthetic probe
cacagtttatcctggagcaccgagccaag

SEQ ID NO: 205 - DNA, 25 bp, Artificial Sequence
  Description of Artificial Sequence Synthetic primer
gaagataaacgactggacggagatt

SEQ ID NO: 206 - DNA, 26 bp, Artificial Sequence
  Description of Artificial Sequence Synthetic primer
aagataaacgactggacggagattac

SEQ ID NO: 207 - DNA, 28 bp, Artificial Sequence
  Description of Artificial Sequence Synthetic primer
acagtttatcctggagcaccgagccaag

SEQ ID NO: 208 - DNA, 26 bp, Artificial Sequence
  Description of Artificial Sequence Synthetic primer
gaagataaacgactggacggagatta

SEQ ID NO: 209 - DNA, 22 bp, Artificial Sequence
  Description of Artificial Sequence Synthetic primer
tccgtccagtcgtttatcttca

SEQ ID NO: 210 - DNA, 18 bp, Artificial Sequence
  Description of Artificial Sequence Synthetic primer
gccaaggcgaccagacaa

SEQ ID NO: 211 - DNA, 23 bp, Artificial Sequence
  Description of Artificial Sequence Synthetic primer
tccgtccagtcgtttatcttcac

SEQ ID NO: 212 - DNA, 29 bp, Artificial Sequence
  Description of Artificial Sequence Synthetic primer
cctgctctagatatcctctttatcatcag

SEQ ID NO: 213 - DNA, 20 bp, Artificial Sequence
  Description of Artificial Sequence Synthetic probe
accaccatccacacggcggc

SEQ ID NO: 214 - DNA, 18 bp, Artificial Sequence
  Description of Artificial Sequence Synthetic primer
gcgcgtgtaccacatcca

SEQ ID NO: 215 - DNA, 25 bp, Artificial Sequence
  Description of Artificial Sequence Synthetic probe
ccccagcctcccgatcacggtttac

SEQ ID NO: 216 - DNA, 18 bp, Artificial Sequence
  Description of Artificial Sequence Synthetic primer
ccgacggtgcgtttagga

SEQ ID NO: 217 - DNA, 19 bp, Artificial Sequence
  Description of Artificial Sequence Synthetic primer
gcctcccgatcacggttta

SEQ ID NO: 218 - DNA, 23 bp, Artificial Sequence
  Description of Artificial Sequence Synthetic probe
cgcagcgtgctcctaaacgcacc

SEQ ID NO: 219 - DNA, 21 bp, Artificial Sequence
  Description of Artificial Sequence Synthetic probe
cctgccgcagcgtgctcctaa

SEQ ID NO: 220 - DNA, 23 bp, Artificial Sequence
  Description of Artificial Sequence Synthetic primer
cttcgaggaggtgatcgacaaga

SEQ ID NO: 221 - DNA, 20 bp, Artificial Sequence
  Description of Artificial Sequence Synthetic primer
ccgcgaggggttgtacttga

SEQ ID NO: 222 - DNA, 18 bp, Artificial Sequence
  Description of Artificial Sequence Synthetic primer
gagaccaccgcgtttcac

SEQ ID NO: 223 - DNA, 28 bp, Artificial Sequence
  Description of Artificial Sequence Synthetic probe
tggcacaccaccgacctcaagtacaacc

SEQ ID NO: 224 - DNA, 29 bp, Artificial Sequence
  Description of Artificial Sequence Synthetic probe
cacaccaccgacctcaagtacaacccctc

SEQ ID NO: 225 - DNA, 26 bp, Artificial Sequence
  Description of Artificial Sequence Synthetic primer
ccaccgacctcaagtacaacccctcg

SEQ ID NO: 226 - DNA, 21 bp, Artificial Sequence
  Description of Artificial Sequence Synthetic primer
aaacggggacatgtacacaaa

(19) United States
(12) Patent Application Publication
(10) Pub. No.: US 2011/0014598 A1
(43) Pub. Date: Jan. 20, 2011

(54) OPTIMIZED PROBES AND PRIMERS AND METHOD OF USING SAME FOR THE DETECTION OF HERPES SIMPLEX VIRUS

(75) Inventors: Heather L.B. Kiefer, Westford, MA (US); James R. Hully, Salisbury, MA (US); William Lauzier, East Bridgewater, MA (US)

Correspondence Address: FOLEY & LARDNER LLP, 111 HUNTINGTON AVENUE, 26TH FLOOR, BOSTON, MA, 02199-7610, US

(73) Assignee: INTELLIGENT MEDICAL DEVICES, INC

(21) Appl. No.: 12/811,984

(22) PCT Filed: Jan. 7, 2009
(86) PCT No.: PCT/US09/30285

§ 371 Date: Jul. 21, 2010

Related U.S. Application Data
(60) Provisional application No. 61/019,537, filed on Jan. 7, 2008.

(51) Int. Cl. C12Q 1/70; C07H 21/04

(52) U.S. Cl. 435/5; 536/24.32; 536/24.33

(57) ABSTRACT

Described herein are primers and probes useful for detecting and typing variant HSV strains, and methods of using the described primers and probes.

### FIELD OF THE INVENTION

The present invention relates to nucleic acid probes and primers for detecting viral genetic material from Herpes simplex virus (HSV) (Type 1 and Type 2).

### BACKGROUND OF THE INVENTION

HSV causes a variety of clinical manifestations at diverse anatomical sites in both normal and immunocompromised patients. Generalized or disseminated HSV infection can cause severe morbidity and mortality in immunologically compromised individuals or through neonatal infection. One such infection causes herpes simplex encephalitis (HSE) is one of the most devastating infections of the central nervous system, and often involves children and adolescents. Almost all HSE cases in adults and children are due to HSV Type 1 (HSV-1) infections, whilst HSV Type 2 (HSV-2) infections are typically associated with neonatal HSE and infections of immunocompromised individuals.

Early detection and subsequent antiviral therapy can have a significant impact on improved patient outcome. Several methods are currently available for detecting HSV, including cell culture and nucleic acid testing, e.g., real-time PCR, but such methods do not adequately address the broad genetic diversity of target HSV and HSE pathogens.

### SUMMARY

In one embodiment, the present invention is directed to an isolated polynucleotide, comprising a nucleotide sequence that comprises any one of SEQ ID NOs: 1-87.

In another embodiment, the present invention is directed to an isolated polynucleotide, comprising any of the nucleotide sequences depicted in Table 3 or any of the nucleotide sequences depicted in Table 4.

In another embodiment, the present invention is directed to a primer pair for amplifying herpes simplex virus DNA, comprising a forward and reverse primer selected from the group consisting of the sequences listed in groups 1-54 of Table 3.

In another embodiment, the present invention is directed to a primer pair for amplifying herpes simplex virus DNA, comprising the forward and reverse primer pairs depicted in Table 4.

In another embodiment, the present invention is directed to a primer pair for amplifying herpes simplex virus DNA selected from the group consisting of (1) SEQ ID NOs: 4 and 10; (2) SEQ ID NOs: 20 and 52; (3) SEQ ID NOs: 70 and 72; (4) SEQ ID NOs: 73 and 75; (5) SEQ ID NOs: 76 and 78; (6) SEQ ID NOs: 79 and 81; (7) SEQ ID NOs: 82 and 78; (8) SEQ ID NOs: 79 and 81; (9) SEQ ID NOs: 83 and 85; and (10) SEQ ID NOs: 79 and 87. In a particular embodiment, the present invention is directed to a polynucleotide probe that binds to a product amplified by one or more of the primer sets described herein, wherein (1) the probe comprising the sequence of SEQ ID NO: 69 hybridizes to the PCR product amplified by SEQ ID NOs: 4 and 10; (2) the probe comprising the sequence of SEQ ID NO: 21 hybridizes to the PCR product amplified by SEQ ID NOs: 20 and 52; (3) the probe comprising the sequence of SEQ ID NO: 71 hybridizes to the PCR product amplified by SEQ ID NOs: 70 and 72; (4) the probe comprising the sequence of SEQ ID NO: 74 hybridizes to the PCR product amplified by SEQ ID NOs: 73 and 75; (5) the probe comprising the sequence of SEQ ID NO: 77 hybridizes to the PCR product amplified by (i) SEQ ID NOs: 76 and 78, and (ii) SEQ ID NOs: 82 and 78; (6) the probe comprising the sequence of SEQ ID NO: 80 hybridizes to the PCR product amplified by SEQ ID NOs: 79 and 81; (7) the probe comprising the sequence of SEQ ID NO: 84 hybridizes to the PCR product amplified by SEQ ID NOs: 83 and 85; (8) the probe comprising the sequence of SEQ ID NO: 84 hybridizes to the PCR product amplified by SEQ ID NOs: 83 and 85; and (9) the probe comprising the sequence of SEQ ID NO: 86 hybridizes to the PCR product amplified by SEQ ID NOs: 79 and 87. In a particular embodiment, the probe is labeled, e.g., the probe comprises a fluorescent label, a chemiluminescent label, a radioactive label, biotin, or gold.

In another embodiment, the present invention is directed to a method for detecting an HSV virus in a sample, comprising (1) adding together at least once group of forward and reverse primers depicted in Tables 3 or 4 to a sample, (2) conducting a polymerase chain reaction on the sample, and (3) detecting the generation of a PCR product, wherein the generation of an amplified PCR product indicates the presence of an HSV variant in the sample. In a particular embodiment, the forward and reverse primers comprise at least one sequence from the group consisting of: (1) SEQ ID NOs: 4 and 10; (2) SEQ ID NOs: 20 and 52; (3) SEQ ID NOs: 70 and 72; (4) SEQ ID NOs: 73 and 75; (5) SEQ ID NOs: 76 and 78; (6) SEQ ID NOs: 79 and 81; (7) SEQ ID NOs: 82 and 78; (8) SEQ ID NOs: 79 and 81; (9) SEQ ID NOs: 83 and 85; and (10) SEQ ID NOs: 79 and 87, respectively. In a particular embodiment, the method further comprises the steps of (1) adding a labeled probe to the sample, wherein the probe comprises the sequence that corresponds to the forward and reverse primer pair group depicted in Tables 3 or 4, and (2) detecting the binding of the probe to an amplified PCR product after exposing the PCR product and probe(s) to conditions that promote hybridization. In a particular embodiment, the sequence of the probe or probes is selected from the group consisting of SEQ ID NOs: 21, 69, 71, 74, 77, 80, 84, and 86. in a particular embodiment, the probe is fluorescently labeled and the step of detecting the binding of the probe to the amplified PCR product entails measuring the fluorescence of the sample. In a particular embodiment, the sample is blood, serum, plasma, sputum, urine, stool, skin, cerebrospinal fluid, saliva, gastric secretions, tears, oropharyngeal swabs, nasopharyngeal swabs, throat swabs, nasal aspirates, nasal wash, and fluids collected from the ear, eye, mouth, respiratory airways, spinal tissue or fluid, cerebral fluid, trigeminal ganglion sample, or a sacral ganglion sample.

### DETAILED DESCRIPTION

The present invention provides nucleic acid primers and probes for detecting and typing viral genetic material, especially HSV viruses, including either or both of Types 1 and 2, and methods for designing and optimizing the respective primer and probe sequences that are useful for detecting and/or typing those HSV viruses. The present invention also therefore provides a method for designing primer and probe sequences that specifically detect the presence of any or specific HSV virus(es) in a given sample. Of particular interest in this regard is the ability of the disclosed primers and probes—as well as those that can be designed according to the disclosed methods—to specifically detect strains and variants of the HSV viruses regardless of Type 1 or 2 variant-specific genomic mutations. The optimized primers and probes of the invention are useful, therefore, for identifying and diagnosing the causative or contributing agents of HSV infection whereupon an appropriate treatment can then be administered to the individual and steps taken to eradicate the virus.

The present invention provides a robust bioinformatic analytical system that is useful for performing a comprehensive analysis of all known target sequences to design primers and probes with the best possible sensitivity and specificity. That is, the primers and probes of the present invention are useful for detecting both types of HSV, HSV-1 and HSV-2, in a singleplex, “non-typing” format that does not necessarily distinguish between HSV-1 and HSV-2; or for detecting and identify both types of HSV in a multiplex, “typing” format.

According to the present invention all HSV Type 1 and Type 2 nucleotide sequences available in GenBank were aligned and regions of conservation were identified based on comparison to various input target genes. The input target that detects both types without discriminating between Type 1 and Type 2 is the HSV Glycoprotein B gene, where analysis of over 60 sequences confirmed the conservation in sequence between both viral types. See Table 1 below. The input targets that detect one of the two types and can discriminate between the two were the HSV-1 Glycoprotein D genes (210 sequences analyzed) and the HSV-2 Glycoprotein G genes (69 sequences analyzed).

TABLE 1

| To detect . . . | then design primers and probes to . . . |
|---|---|
| HSV-1 and HSV-2 | the HSV Glycoprotein B gene |
| HSV-1 alone | the HSV-1 Glycoprotein D gene |
| HSV-2 alone | the HSV-2 Glycoprotein G gene |

These targets were chosen because they are well conserved between the two HSV types, but have enough variability to allow specificity between them, and have substantial sequence information publicly available from multiple HSV strains and geographic regions.

Thus the present invention provides one or more pairs of PCR primers that can anneal to HSV variants and thereby amplify a PCR product from a biological sample. Hence, the present invention provides a first PCR primer and a second PCR primer, each of which comprises a nucleotide sequence designed according to the inventive principles disclosed herein, which are used together to positively identify the presence of HSV in a sample regardless of the actual nucleotide composition of the infecting HSV variant(s). The generation of an amplified PCR product or products from a sample using the primer pairs disclosed herein is diagnostically indicative of an HSV infection or at least indicative of the presence of an HSV variant in the sample. Of note, each of the primer sequences can be used as probes to detect viral variants.

Also provided by the present invention are probes that hybridize to amplified PCR products or unamplified sample sequences. A probe can be labeled, for example, such that when it binds to an internal PCR product target sequence, or after it has been cleaved after binding, a fluorescent signal is emitted that is detectable under various spectroscopy and light-measurement apparatuses. The use of a labeled probe, therefore, can enhance the specificity of the PCR-based amplification of variant HSV DNA because it permits the detection of virus DNA at low template concentrations that might not be conducive to visual detection as a gel-stained PCR product.

Primers and probes of the invention are sequences that anneal to a viral genomie sequence, e.g., HSV (the “target”). The target sequence can be, for example, a viral genome or a subset, “region”, of a viral genome. In one embodiment, the entire genomic sequence can be “scanned” for optimized primers and probes useful for detecting viral variants. In other embodiments, particular regions of the viral genome can be scanned, e.g., regions that are documented in the literature as being useful for detecting multiple variants, regions that are conserved, or regions where sufficient information is available in, for example, a public database, with respect to viral variants.

Sets of primers and probes are generated based on the target to be detected. The set of all possible primers and probes can include, for example, sequences that include the variability at every site based on the known viral variants, or the primers and probes can be generated based on a consensus sequence of the target. The primers and probes are generated such that the primers and probes are able to anneal to a particular variant or a consensus sequence under high stringency conditions. For example, one of skill in the art recognizes that for any particular sequence, it is possible to provide more than one oligonucleotide sequence that will anneal to the particular target sequence, even under high stringency conditions. The set of primers and probes to be sampled for the purposes of the present invention includes, for example, all such oligonucleotides for all viral variant sequence. Alternatively, the primers and probes includes all such oligonucleotides for a given consensus sequence for a target.

Typically, stringent hybridization and washing conditions are used for nucleic acid molecules over about 500 bp. Stringent hybridization conditions include a solution comprising about 1 M Na⁺ at 25° C. to 30° C. below the Tm; e.g., 5×SSPE, 0.5% SDS, at 65° C.; see, Ausubel, et al., Current Protocols in Molecular Biology, Greene Publishing, 1995; Sambrook et al., Molecular Cloning: A Laboratory Manual, Cold Spring Harbor Press, 1989). Tm is dependent on both the G+C content and the concentration of Na⁺. A formula to calculate the Tm of nucleic acid molecules greater than about 500 by is Tm=81.5+0.41(%(G+C)) log₁₀[Na⁺]. Washing conditions are generally performed at least at equivalent stringency conditions as the hybridization. If the background levels are high, washing can be performed at higher stringency, such as around 15° C. below the Tm.

The set of primers and probes, once determined as described above, are optimized for hybridizing to a plurality of viral variants by employing scoring and/or ranking steps that provide a positive or negative preference or “weight” to certain nucleotides in a target nucleic acid variant sequence. For example, if a consensus sequence is used to generate the full set of primers and probes, then a particular primer sequence is scored for its ability to anneal to the corresponding sequence of every known native variant sequence. Even if a probe was originally generated based on a consensus, therefore, the validation of the probe is in its ability to specifically anneal and detect every or a large majority of variant viral sequences. The particular scoring or ranking steps performed depend upon the intended use for the primer and/or probe, the particular target nucleic acid sequence, and the number of variants of that target nucleic acid sequence. The methods of the invention provide optimal primer and probe sequences because they hybridize to all or a subset of HSV variants. Once optimized oligonucleotides are identified that can anneal to viral variants, the sequences can then further be optimized for use, for example, in conjunction with another optimized sequence as a “primer pair” or for use as a probe.

Primer or probe sequences can be ranked according to specific hybridization parameters or metrics that assign a score value indicating their ability to anneal to viral variants under highly stringent conditions. Where a primer pair is being scored, a “first” or “forward” primer is scored and the “second” or “reverse”-oriented primer sequences can be optimized similarly, followed by an optional evaluation for cross-reactivity, for example, between the forward and reverse primers.

The scoring or ranking steps that are used in the methods of the invention include, for example, the following parameters: a target sequence score for the target nucleic acid sequence(s), e.g., the PriMD® score; a mean conservation score for the target nucleic acid sequence(s); a mean coverage score for the target nucleic acid sequence(s); 100% conservation score of a portion (e.g., 5′ end, center, 3′ end) of the target nucleic acid sequence(s); a species score; a strain score; a subtype score; a serotype score; an associated disease score; a year score; a country of origin score; a duplicate score; a patent score; and a minimum qualifying score. Other parameters that are used include, for example, the number of mismatches, the number of critical mismatches (e.g., mismatches that result in the predicted failure of the sequence to anneal to a target sequence), the number of native variant sequences that contain critical mismatches, and predicted Tm values. The term “Tm” means the temperature at which a population of double-stranded nucleic acid molecules becomes half-dissociated into single strands. Methods for calculating the Tm of nucleic acids are well known in the art (Berger and Kimmel (1987) Meth, Enzymol., Vol. 152: Guide To Molecular Cloning Techniques, San Diego: Academic Press, Inc. and Sambrook et al. (1989) Molecular Cloning: A Laboratory Manual, (2nd ed.) Vols. 1-3, Cold Spring Harbor Laboratory).

The resultant scores represent steps in determining nucleotide or whole target nucleic acid sequence preference, while tailoring the primer and/or probe sequences so that they hybridize to a plurality of target nucleic acid variants. The methods of the invention also can comprise the step of allowing for one or more nucleotide changes when determining identity between the candidate primer and probe sequences and the target nucleic acid variant sequences, or their complements.

In another embodiment, the methods of the invention comprise the steps of comparing the candidate primer and probe nucleic acid sequences to “exclusion nucleic acid sequences” and then rejecting those candidate nucleic acid sequences that share identity with the exclusion nucleic acid sequences. In another embodiment, the methods of the invention comprise the steps of comparing the candidate primer and probe nucleic acid sequences to “inclusion nucleic acid sequences” and then rejecting those candidate nucleic acid sequences that do not share identity with the inclusion nucleic acid sequences.

A target nucleotide sequence of the present invention from which the primers and probes are designed, can be the entire HSV genome, a region thereof, or any HSV gene. In one aspect of the present invention the target gene is an HSV glycoprotein gene, such as glycoprotein B, D, or G. Accordingly, the present invention provides primers and probes that, for example, can amplify or detect all or glycoprotein B, D, or G protein variant sequences. The set of primers and probes described herein were generated based on a consensus matrix sequence, and then optimized against all known viral variants.

In an embodiment of the methods of the invention, optimizing primers and probes comprises using a polymerase chain reaction (PCR) penalty score formula comprising at least one of a weighted sum of: primer Tm—optimal Tm; difference between primer Tms; amplicon length—minimum amplicon length; and distance between the primer and a TaqMan® probe. The optimizing step also can comprise determining the ability of the candidate sequence to hybridize with the most target nucleic acid variant sequences (e.g., the most target organisms or genes). In another embodiment, the selecting or optimizing step comprises determining which sequences have mean conservation scores closest to 1, wherein a standard of deviation on the mean conservation scores is also compared.

In other embodiments, the methods further comprise the step of evaluating which target nucleic acid variant sequences are hybridized by an optimal forward primer and an optimal reverse primer, for example, by determining the number of base differences between target nucleic acid variant sequences in a database. For example, the evaluating step can comprise performing an in silico polymerase chain reaction, involving (1) rejecting the forward primer and/or reverse primer if it does not meet inclusion or exclusion criteria; (2) rejecting the forward primer and/or reverse primer if it does not amplify a medically valuable nucleic acid; (3) conducting a BLAST analysis to identify forward primer sequences and/or reverse primer sequences that overlap with a published and/or patented sequence; (4) and/or determining the secondary structure of the forward primer, reverse primer, and/or target. In an embodiment, the evaluating step includes evaluating whether the forward primer sequence, reverse primer sequence, and/or probe sequence hybridizes to sequences in the database other than the nucleic acid sequences that are representative of the target variants.

The present invention provides polynucleotides that have preferred primer and probe qualities. These qualities are specific to the sequences of the optimized probes, however, one of skill in the art would recognize that other molecules with similar sequences could also be used. The oligonucleotides provided herein comprise a sequence that shares at least about 60-70% identity with a sequence described in Table 2. In addition, the sequences can be incorporated into longer sequences, provided they function to specifically anneal to and identify viral variants. In another embodiment, the invention provides a nucleic acid comprising a sequence that shares at least about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity with the sequences disclosed herein or complement thereof. The terms “homology” or “identity” or “similarity” refer to sequence relationships between two nucleic acid molecules and can be determined by comparing a nucleotide position in each sequence when aligned for purposes of comparison. The term “homology” refers to the relatedness of two nucleic acid or protein sequences. The term “identity” refers to the degree to which nucleic acids are the same between two sequences. The term “similarity” refers to the degree to which nucleic acids are the same, but includes neutral degenerate nucleotides that can be substituted within a codon without changing the amino acid identity of the codon, as is well known in the art. The probe and/or primer nucleic acid sequences of the invention are optimal for identifying numerous variants of a target nucleic acid, e.g., from an HSV pathogen. In an embodiment, the nucleic acids of the invention are primers for the synthesis (e.g., amplification) of target nucleic acid variants and/or probes for identification, isolation, detection, or analysis of target nucleic acid variants, e.g., an amplified target nucleic acid variant that is amplified using the primers of the invention.

The present polynucleotides hybridize with more than one viral variants (variants as determined by differences in their genomic sequence). The probes and primers provided herein can, for example, allow for the detection of all known viral variants or a subset thereof. In addition, the primers and probes of the present invention, depending on the variant sequence(s), can allow for the detection of previously unknown viral variants. The methods of the invention provide for optimal primers and probes, and sets thereof, and combinations of sets thereof, which can hybridize with a larger number of target variants than available primers and probes.

In other aspects, the invention also provides vectors (e.g., plasmid, phage, expression), cell lines (e.g., mammalian, insect, yeast, bacterial), and kits comprising any of the sequences of the invention described herein. The invention further provides target nucleic acid variant sequences that are identified, for example, using the methods of the invention. In an embodiment, the target nucleic acid variant sequence is an amplification product. In another embodiment, the target nucleic acid variant sequence is a native or synthetic nucleic acid. The primers, probes, and target nucleic acid variant sequences, vectors, cell lines, and kits can have any number of uses, such as diagnostic, investigative, confirmatory, monitoring, predictive or prognostic.

A diagnostic kit is provided by the present invention that comprises one or more of the polynucleotides described herein, which are useful for detecting and/or typing HSV infection in an individual. An individual can be a human male, human female, human adult, human child, or human fetus. An individual can also be any mammal, reptile, avian, fish, or amphibian. Hence, an individual can be a mouse, rat, sheep, dog, simian, horse, cattle, chicken, porcine, lamb, bird or fish.

A probe of the present invention can comprise a label, such as a fluorescent label, a chemiluminescent label, a radioactive label, biotin, gold, dendrimers, aptamer, enzymes, proteins, and molecular motors. In an embodiment, the probe is a hydrolysis probe, such as, for example, a TaqMan® probe. In other embodiments, the probes of the invention are molecular beacons, SYBR Green® primers, or fluorescence energy transfer (FRET) probes.

Polynucleotides of the present invention do not only include primers that are useful for conducting the aforementioned PCR amplification reactions, but also include polynucleotides that are attached to a solid support, such as, for example, a microarray, multiwell plate, column, bead, glass slide, polymeric membrane, glass microfiber, plastic tubes, cellulose, and carbon nanostructures. Hence, detection of HSV variants can be performed by exposing such a polynucleotide-covered surface to a sample such that the binding of a complementary variant DNA sequence to a surface-attached polynucleotide elicits a detectable signal or reaction.

One embodiment of the invention uses solid support-based oligonucleotide hybridization methods to detect gene expression. Solid support-based methods suitable for practicing the present invention are widely known and are described (PCT application WO 95/11755; Huber et al., Anal. Biochem., 299:24, 2001; Meiyanto et al., Biotechniques, 31:406, 2001; Relogio et al., Nucleic Acids Res., 30:e51, 2002; the contents of which are incorporated herein by reference in their entirety). Any solid surface to which oligonucleotides can be bound, covalently or non-covalently, can be used. Such solid supports include, but are not limited to, filters, polyvinyl chloride dishes, silicon or glass based chips.

In certain embodiments, the nucleic acid molecule can be directly bound to the solid support or bound through a linker arm, which is typically positioned between the nucleic acid sequence and the solid support. A linker arm that increases the distance between the nucleic acid molecule and the substrate can increase hybridization efficiency. There are a number of ways to position a linker arm. In one common approach, the solid support is coated with a polymeric layer that provides linker arms with a plurality of reactive ends/sites. A common example of this type is glass slides coated with polylysine (U.S. Pat. No. 5,667,976, the contents of which are incorporated herein by reference in its entirety), which are commercially available. Alternatively, the linker arm can be synthesized as part of or conjugated to the nucleic acid molecule, and then this complex is bonded to the solid support. One approach, for example, takes advantage of the extremely high affinity biotin-streptavidin interaction. The streptavidin-biotinylated reaction is stable enough to withstand stringent washing conditions and is sufficiently stable that it is not cleaved by laser pulses used in some detection systems, such as matrix-assisted laser desorption/ionization time of flight (MALDI-TOF) mass spectrometry. Therefore, streptavidin can be covalently attached to a solid support, and a biotinylated the nucleic acid molecule will bind to the streptavidin-coated surface. In one version of this method, an amino-coated silicon wafer is reacted with the n-hydroxysuccinimido-ester of biotin and complexed with streptavidin. Biotinylated oligonucleotides are bound to the surface at a concentration of about 20 fmol DNA per mm².

Alternatively, one can directly bind DNA to the support using carbodiimides, for example. In one such method, the support is coated with hydrazide groups, then treated with carbodiimide. Carboxy-modified nucleic acid molecules are then coupled to the treated support. Epoxide-based chemistries are also being employed with amine modified oligonucleotides. Other chemistries for coupling nucleic acid molecules to solid substrates are known to those of skill in the art.

The nucleic acid molecules, e.g., the primers and probes of the present invention, must be delivered to the substrate material, which is suspected of containing or is being tested for the presence of HSV. Because of the miniaturization of the arrays, delivery techniques must be capable of positioning very small amounts of liquids in very small regions, very close to one another and amenable to automation. Several techniques and devices are available to achieve such delivery. Among these are mechanical mechanisms (e.g., arrayers from QeneticMicroSystems, MA, USA) and ink-jet technology. Very fine pipets can also be used.

Other formats are also suitable within the context of this invention. For example, a 96-well format with fixation of the nucleic acids to a nitrocellulose or nylon membrane may also be employed.

After the nucleic acid molecules have been bound to the solid support, it is often useful to block reactive sites on the solid support that are not consumed in binding to the nucleic acid molecule. In the absence of the blocking step, the probes can, to some extent, bind directly to the solid support itself, giving rise to non-specific binding. Non-specific binding can sometimes hinder the ability to detect low levels of specific binding. A variety of effective blocking agents (e.g., milk powder, serum albumin or other proteins with free amine groups, polyvinylpyrrolidine) can be used and others are known to those skilled in the art (U.S. Pat. No. 5,994,065, the contents of which are incorporated herein by reference in their entirety). The choice depends at least in part upon the binding chemistry.

One embodiment uses oligonucleotide arrays, e.g., microarrays, that can be used to simultaneously observe the expression of a number of HSV variant genes, such as the matrix protein gene. Oligonucleotide arrays comprise two or more oligonucleotide probes provided on a solid support, wherein each probe occupies a unique location on the support. The location of each probe may be predetermined, such that detection of a detectable signal at a given location is indicative of hybridization to an oligonucleotide probe of a known identity. Each predetermined location can contain more than one molecule of a probe, but each molecule within the predetermined location has an identical sequence. Such predetermined locations are termed features. There can be, for example, from 2, 10, 100, 1,000, 2,000 or 5,000 or more of such features on a single solid support. In one embodiment, each oligonucleotide is located at a unique position on an array at least 2, at least 3, at least 4, at least 5, at least 6, or at least 10 times.

Oligonucleotide probe arrays for detecting gene expression can be made and used according to conventional techniques described (Lockhart et al., Nat. Biotech., 14:1675-1680, 1996; McGall et al., Proc. Natl. Acad. Sci. USA, 93:13555, 1996; Hughes et al., Nat. Biotechnol., 19:342, 2001). A variety of oligonucleotide array designs is suitable for the practice of this invention.

Generally, a detectable molecule, also referred to herein as a label, can be incorporated or added to an array's probe nucleic acid sequences. Many types of molecules can be used within the context of this invention. Such molecules include, but are not limited to, fluorochromes, chemiluminescent molecules, chromogenic molecules, radioactive molecules, mass spectrometry tags, proteins, and the like. Other labels will be readily apparent to one skilled in the art.

Oligonucleotide probes used in the methods of the present invention, including microarray techniques, can be generated using PCR. PCR primers used in generating the probes are chosen, for example, based on the sequences of Example 4. In one embodiment, oligonucleotide control probes also are used. Exemplary control probes can fall into at least one of three categories referred to herein as (1) normalization controls, (2) expression level controls and (3) negative controls. In microarray methods, one or more of these control probes can be provided on the array with the inventive cell cycle gene-related oligonucleotides.

Normalization controls correct for dye biases, tissue biases, dust, slide irregularities, malformed slide spots, etc. Normalization controls are oligonucleotide or other nucleic acid probes that are complementary to labeled reference oligonucleotides or other nucleic acid sequences that are added to the nucleic acid sample to be screened. The signals obtained from the normalization controls, after hybridization, provide a control for variations in hybridization conditions, label intensity, reading efficiency and other factors that can cause the signal of a perfect hybridization to vary between arrays. In one embodiment, signals (e.g., fluorescence intensity or radioactivity) read from all other probes used in the method are divided by the signal from the control probes, thereby normalizing the measurements.

Virtually any probe can serve as a normalization control. Hybridization efficiency varies, however, with base composition and probe length. Preferred normalization probes are selected to reflect the average length of the other probes being used, but they also can be selected to cover a range of lengths. Further, the normalization control(s) can be selected to reflect the average base composition of the other probe(s) being used. In one embodiment, only one or a few normalization probes are used, and they are selected such that they hybridize well (i.e., without forming secondary structures) and do not match any test probes. In one embodiment, the normalization controls are mammalian genes.

“Negative control” probes are not complementary to any of the test oligonucleotides (i.e., the inventive cell cycle gene-related oligonucleotides), normalization controls, or expression controls. In one embodiment, the negative control is a mammalian gene which is not complementary to any other sequence in the sample.

The terms “background” and “background signal intensity” refer to hybridization signals resulting from non-specific binding or other interactions between the labeled target nucleic acids (e.g., mRNA present in the biological sample) and components of the oligonucleotide array. Background signals also can be produced by intrinsic fluorescence of the array components themselves. A single background signal can be calculated for the entire array, or a different background signal can be calculated for each target nucleic acid. In a one embodiment, background is calculated as the average hybridization signal intensity for the lowest 5 to 10 percent of the oligonucleotide probes being used, or, where a different background signal is calculated for each target gene, for the lowest 5 to 10 percent of the probes for each gene. Where the oligonucleotide probes corresponding to a particular HSV target hybridize well and, hence, appear to bind specifically to a target sequence, they should not be used in a background signal calculation. Alternatively, background can be calculated as the average hybridization signal intensity produced by hybridization to probes that are not complementary to any sequence found in the sample (e.g., probes directed to nucleic acids of the opposite sense or to genes not found in the sample). In microarray methods, background can be calculated as the average signal intensity produced by regions of the array that lack any oligonucleotides probes at all.

In an alternative embodiment, the nucleic acid molecules are directly or indirectly coupled to an enzyme. Following hybridization, a chromogenic substrate is applied and the colored product is detected by a camera, such as a charge-coupled camera. Examples of such enzymes include alkaline phosphatase, horseradish peroxidase and the like. The invention also provides methods of labeling nucleic acid molecules with cleavable mass spectrometry tags (CMST; U.S. Pat. No. 60/279,890). After an assay is complete, and the uniquely CMST-labeled probes are distributed across the array, a laser beam is sequentially directed to each member of the array. The light from the laser beam both cleaves the unique tag from the tag-nucleic acid molecule conjugate and volatilizes it. The volatilized tag is directed into a mass spectrometer. Based on the mass spectrum of the tag and knowledge of how the tagged nucleotides were prepared, one can unambiguously identify the nucleic acid molecules to which the tag was attached (WO 9905319).

The nucleic acids, primers and probes of the present invention can be labeled readily by any of a variety of techniques. When the diversity panel is generated by amplification, the nucleic acids can be labeled during the reaction by incorporation of a labeled dNTP or use of labeled amplification primer. If the amplification primers include a promoter for an RNA polymerase, a post-reaction labeling can be achieved by synthesizing RNA in the presence of labeled NTPs. Amplified fragments that were unlabeled during amplification or unamplified nucleic acid molecules can be labeled by one of a number of end labeling techniques or by a transcription method, such as nick-translation, random-primed DNA synthesis. Details of these methods are known to one of skill in the art and are set out in methodology books. Other types of labeling reactions are performed by denaturation of the nucleic acid molecules in the presence of a DNA-binding molecule, such as RecA, and subsequent hybridization under conditions that favor the formation of a stable RecA-incorporated DNA complex.

In another embodiment, PCR-based methods are used to detect gene expression. These methods include reverse-transcriptase-mediated polymerase chain reaction (RT-PCR) including real-time and endpoint quantitative reverse-transcriptase-mediated polymerase chain reaction (Q-RTPCR). These methods are well known in the art. For example, methods of quantitative PCR can be carried out using kits and methods that are commercially available from, for example, Applied BioSystems and Stratagene®. See also Kochanowski, Quantitative PCR Protocols (Humana Press, 1999); Innis et al., supra.; Vandesompele et al., Genome Biol., 3:RESEARCH0034, 2002; Stein, Cell Mol. Life Sci. 59:1235, 2002.

The forward and reverse amplification primers and internal hybridization probe is designed to hybridize specifically and uniquely with one nucleotide derived from the transcript of a target gene. In one embodiment, the selection criteria for primer and probe sequences incorporates constraints regarding nucleotide content and size to accommodate TaqMan® requirements. SYBR Green® can be used as a probe-less Q-RTPCR alternative to the TaqMan®-type assay, discussed above (ABI Prism® 7900 Sequence Detection System User Guide Applied Biosystems, chap. 1-8, App. A-F. (2002)). A device measures changes in fluorescence emission intensity during PCR amplification. The measurement is done in “real time,” that is, as the amplification product accumulates in the reaction. Other methods can be used to measure changes in fluorescence resulting from probe digestion. For example, fluorescence polarization can distinguish between large and small molecules based on molecular tumbling (U.S. Pat. No. 5,593,867).

The primers and probes of the present invention may anneal to or hybridize to various HSV genetic material or genetic material derived therefrom, such as RNA, DNA, cDNA, or a PCR product.

A “sample” that is tested for the presence of an HSV variant includes, but is not limited to a tissue sample, such as, for example, blood, serum, plasma, sputum, urine, stool, skin, cerebrospinal fluid, saliva, gastric secretions, hair, and tear fluid. A sample can be obtained by an oropharyngeal swab, nasopharyngeal swab, throat swab, nasal aspirate, nasal wash, or fluid collected from the ear, eye, mouth, or respiratory airway. The tissue sample may be fresh, fixed, preserved, or frozen. A sample also includes any item, surface, material, or clothing, or environment in which it may be desirable to test for the presence of HSV variant(s). Thus, for instance, the present invention includes testing door handles, faucets, table surfaces, elevator buttons, chairs, toilet seats, sinks, kitchen surfaces, children's cribs, bed linen, pillows, keyboards, and so on, for the presence of HSV variants.

The target nucleic acid variant that is amplified may be RNA or DNA or a modification thereof. Thus, the amplifying step man comprise isothermal or non-isothermal reaction such as polymerase chain reaction, Scorpion® primers, molecular beacons, SimpleProbes®, HyBeacons®, cycling probe technology, Invader Assay, self-sustained sequence replication, nucleic acid sequence-based amplification, ramification amplifying method, hybridization signal amplification method, rolling circle amplification, multiple displacement amplification, thermophilic strand displacement amplification, transcription-mediated amplification, ligase chain reaction, signal mediated amplification of RNA, split promoter amplification, Q-Beta replicase, isothermal chain reaction, one cut event amplification, loop-mediated isotheiuial amplification, molecular inversion probes, ampliprobe, headloop DNA amplification, and ligation activated transcription. The amplifying step can be conducted on a solid support, such as a multiwell plate, array, column, bead, glass slide, polymeric membrane, glass microfiber, plastic tubes, cellulose, and carbon nanostructures. The amplifying step also comprises in situ hybridization. The detecting step can comprise gel electrophoresis, fluorescence resonant energy transfer, or hybridization to a labeled probe, such as a probe labeled with biotin, at least one fluorescent moiety, an antigen, a molecular weight tag, and a modifier of probe Tm. The detecting step comprises measuring fluorescence, mass, charge, and/or chemiluminescence.

Hybridization may be detected in a variety of ways and with a variety of equipment. In general, the methods can be categorized as those that rely upon detectable molecules incorporated into the diversity panels and those that rely upon measurable properties of double-stranded nucleic acids (e.g., hybridized nucleic acids) that distinguish them from single-stranded nucleic acids (e.g., unhybridized nucleic acids). The latter category of methods includes intercalation of dyes, such as, for example, ethidium bromide, into double-stranded nucleic acids, differential absorbance properties of double and single stranded nucleic acids, binding of proteins that preferentially bind double-stranded nucleic acids, and the like.

### EXEMPLIFICATION

### Example 1

### Scoring a Set of Predicted Annealing Oligonucleotides

Each of the sets of primers and probes selected can be ranked by a combination of methods as individual primers and probes and as a primer/probe set. This will involve one or more method of ranking (e.g., joint ranking, hierarchical ranking, and serial ranking) where sets of primers and probes are eliminated or included based on any combination of the following criteria, and a weighted ranking again based on any combination of the following criteria, for example: (A) Percentage Identity to Target Variants; (B) Conservation Score; (C) Coverage Score; (D) Strain/Subtype/Serotype Score; (E) Associated Disease Score; (F) Duplicates Sequences Score; (G) Year and Country of Origin Score; (H) patent Score, and (I) Epidemiology Score.

(A) Percentage Identity

A percentage identity score is based upon the number of target nucleic acid variant (e.g., native) sequences that can hybridize with perfect conservation (the sequences are perfectly complimentary) to each primer or probe of a primer pair and probe set. If the score is less than 100%, the program ranks additional primer pair and probe sets that are not perfectly conserved. This is a hierarchical scale for percent identity starting with perfect complimentarily, then one base degeneracy through to the number of degenerate bases that would provide the score closest to 100%. The position of these degenerate bases would then be ranked. The methods for calculating the conservation is described under section B.

(i) Individual Base Conservation Score

A set of conservation scores is generated for each nucleotide base in the consensus sequence and these scores represent how many of the target nucleic acid variants sequences have a particular base at this position. For example, a score of 0.95 for a nucleotide with an adenosine, and 0.05 for a nucleotide with a cytidine means that 95% of the native sequences have an A at that position and 5% have a C at that position. A perfectly conserved base position is one where all the target nucleic acid variant sequences have the same base (either an A, C, G, or T/U) at that position. If there is an equal number of bases (e.g., 50% A & 50% T) at a position, it is identified with an N.

(ii) Candidate Primer/Probe Sequence Conservation

An overall conservation score is generated for each candidate primer or probe sequence that represents how many of the target nucleic acid variant sequences will hybridize to the primers or probes. A candidate sequence that is perfectly complimentary to all the target nucleic acid variant sequences will have a score of 1.0 and rank the highest. For example, illustrated below in Table 2 are three different 10-base candidate probe sequences that are targeted to different regions of a consensus target nucleic acid variant sequence. Each candidate probe sequence is compared to a total of 10 native sequences.

TABLE 2

| #1. | A | A | A | C | A | C | G | T | G | C |
|---|---|---|---|---|---|---|---|---|---|---|
| | 0.7 | 1.0 | 1.0 | 1.0 | 1.0 | 1.0 | 1.0 | 1.0 | 1.0 | 1.0 |

→Number of target nucleic acid variant sequences that are perfectly
complimentary - 7. Three out of the ten sequences do not have an A
at position 1.

| #2. | C | C | T | T | G | T | T | C | C | A |
|---|---|---|---|---|---|---|---|---|---|---|
| | 1.0 | 0.9 | 1.0 | 0.9 | 0.9 | 1.0 | 1.0 | 1.0 | 1.0 | 1.0 |

→Number of target nucleic acid variant sequences that are perfectly
complimentary - 7, 8, or 9. At least one target nucleic acid
variant does not have a C at position 2, T at position 4, or G at
position 5. These differences may all be on one target nucleic acid
variant molecule or may be on two or three separate molecules.

| #3. | C | A | G | G | G | A | C | G | A | T |
|---|---|---|---|---|---|---|---|---|---|---|
| | 1.0 | 1.0 | 1.0 | 1.0 | 1.0 | 0.9 | 0.8 | 1.0 | 1.0 | 1.0 |

→Number of target nucleic acid variant sequences that are perfectly
complimentary - 7 or 8. At least one target nucleic acid variant
does not have an A at position 6 and at least two target nucleic
acid variant do not have a C at position 7. These differences may
all be on one target nucleic acid variant molecule or may be on two
separate molecules.

A simple arithmetic mean for each candidate sequence would generate the same value of 0.985. However, the number of target nucleic acid variant sequences identified by each candidate probe sequence can be very different. Sequence #1 can only identify 7 native sequences because of the 0.7 (out of 1.0) score by the first base—A. Sequence #2 has three bases each with a score of 0.9; each of these could represent a different or shared target nucleic acid variant sequence. Consequently, Sequence #2 can identify 7, 8 or 9 target nucleic acid variant sequences. Similarly, Sequence #3 can identify 7 or 8 of the target nucleic acid variant sequences. Therefore, Sequence #2 would be the best choice if all the three bases with a score of 0.9 represented the same 9 target nucleic acid variant sequences.

(iii) Overall Conservation Score of the Primer and Probe Set—Percent Identity

The same method described in (ii) when applied to the complete primer pair and probe set will generate the percent identity for the set (see A above). For example, using the same sequences illustrated above, if Sequences #1 and #2 are primers and Sequence #3 is a probe, then the percent identity for the target can be calculated from how many of the target nucleic acid variant sequences are identified with perfect complimentarity by all three primer/probe sequences. The percent identity could be no better than 0.7 (7 out of 10 target nucleic acid variant sequences) but as little as 0.1 if each of the degenerate bases reflects a different target nucleic acid variant sequence. Again, an arithmetic mean of these three sequences would be 0.985. As none of the above examples were able to capture all the target nucleic acid variant sequences because of the degeneracy (scores of less than 1.0), the ranking system takes into account that a certain amount of degeneracy can be tolerated under normal hybridization conditions, for example, during a polymerase chain reaction. The ranking of these degeneracies is described in (iv) below.

An in silico evaluation determines how many native sequences (e.g., original sequences submitted to public databases) are identified by a given candidate primer/probe set. The ideal candidate primer/probe set is one that can perform PCR and the sequences are perfectly complimentary to all the known native sequences that were used to generate the consensus sequence. If there is no such candidate, then the sets are ranked according to how many degenerate bases can be accepted and still hybridize to just the target sequence during the PCR and yet identify all the native sequences.

The hybridization conditions, for TaqMan® as an example are: 10-50 mM Tris-HCl pH 8.3, 50 mM KCl, 0.1-0.2% Triton® X-100 or 0.1% Tween®, 1-5 mM MgCl₂. The hybridization is performed at 58-60° C. for the primers and 68-70° C. for the probe. The in silico PCR identifies native sequences that are not amplifiable using the candidate primers and probe set. The rules can be as simple as counting the number of degenerate bases to more sophisticated approaches based on exploiting the PCR criteria used by the PriMD® software. Each target nucleic acid variant sequence has a value or weight (see Score assignment above). If the failed target nucleic acid variant sequence is medically valuable, the primer/probe set is rejected. This in silico analysis provides a degree of confidence for a given genotype and is important when new sequences are added to the databases. New target nucleic acid variant sequences are automatically entered into both the “include” and “exclude” categories, For example, a new HSV sequence is tested against an HSV primer/probe set of the invention in the include category but will be added to the exclude category when it is tested against other primer/probe sets, such as HSV. Published primer and probes will also be ranked by the PriMD software.

(iv) Position (5′ to 3′) of the Base Conservation Score

In an embodiment, primers do not have bases in the terminal five positions at the 3′ end with a score less than 1. This is one of the last parameters to be relaxed if the method fails to select any candidate sequences. The next best candidate having a perfectly conserved primer would be one where the poorer conserved positions are limited to the terminal bases at the 5′ end. The closer the poorer conserved position is to the 5′ end, the better the score. For probes, the position criteria is different. For example, with a TaqMan® probe, the most destabilizing effect occurs in the center of the probe. The 5′ end of the probe is also important as this contains the reporter molecule that must be cleaved, following hybridization to the target, by the polymerase to generate a sequence-specific signal. The 3′ end is less critical. Therefore, a sequence with a perfectly conserved middle region will have the higher score. The remaining ends of the probe are ranked in a similar fashion to the 5′ end of the primer. Thus, the next best candidate to a perfectly conserved TaqMan® probe would be one where the poorer conserved positions are limited to the terminal bases at either the 5′ or 3′ ends. The hierarchical scoring will select primers with only one degeneracy first, then primers with two degeneracies next and so on. The relative position of each degeneracy will then be ranked favoring those that are closest to the 5′ end of the primers and those closest to the 3′ end of the TaqMan® probe. If there are two or more degenerate bases in a primer and probe set the ranking will initially select the sets where the degeneracies occur on different sequences.

B. Coverage Score

The total number of aligned sequences is considered under coverage score. A value is assigned to each position based on how many times that position has been reported or sequenced. Alternatively, coverage can be defined as how representative the sequences are of the known strains, subtypes etc., or their relevance to a certain diseases. For example, the target nucleic acid variant sequences for a particular gene may be very well conserved and show complete coverage but certain strains are not represented in those sequences.

A sequence is included if it aligns with any part of the consensus sequence, which is usually a whole gene or a functional unit, or has been described as being a representative of this gene. Even though a base position is perfectly conserved it may only represent a fraction of the total number of sequences (for example, if there are very few sequences). For example, region A of a gene shows a 100% conservation from 20 sequence entries while region B in the same gene shows a 98% conservation but from 200 sequence entries. There is a relationship between conservation and coverage if the sequence shows some persistent variability. As more sequences are aligned, the conservation score falls, but this effect is lessened as the number of sequences gets larger. Unless the number of sequences is very small (e.g., under 10) the value of the coverage score is small compared to that of the conservation score. To obtain the best consensus sequence, artificial spaces are allowed to be introduced. Such spaces are not considered in the coverage score.

D. Strain/Subtype/Serotype Score

A value is assigned to each strain or subtype or serotype based upon its relevance to a disease. For example, strains of INF-A that are linked to pandemics will have a higher score than strains that are generally regarded as benign or included in the current vaccine. The score is based upon sufficient evidence to automatically associate a particular strain with a disease. For example, certain strains of adenovirus are not associated with diseases of the upper respiratory system. Accordingly, there will be sequences included in the consensus sequence that are not associated with diseases of the upper respiratory system.

E. Associated Disease Score

The associated disease score pertains to strains that are not known to be associated with a particular disease (to differentiate from D above). Here, a value is assigned only if the submitted sequence is directly linked to the disease and that disease is pertinent to the assay.

F. Duplicate Sequences Score

If a particular sequence has been sequenced more than once it will have an effect on representation, for example, a strain that is represented by 12 entries in GenBank of which six are identical and the other six are unique. Unless the identical sequences can be assigned to different strains/subtypes (usually by sequencing other gene or by immunology methods) they will be excluded from the scoring.

G. Year and Country of Origin Score

The year and country of origin scores are important in terms of the age of the human population and the need to provide a product for a global market. For example, strains identified or collected many years ago may not be relevant today. Furthermore, it is probably difficult to obtain samples that contain these older strains. In addition, some strains may have the potential for creating an epidemic if most of the present population does not have immunity. Certain divergent strains from more obscure countries or sources may also be less relevant to the locations that will likely perform clinical tests, or may be more important for certain countries (e.g., North America, Europe, or Asia).

H. Patent Score

Candidate target variant sequences published in patents are searched electronically and annotated such that patented regions are excluded. Alternatively, candidate sequences are checked against a patented sequence database.

I. Minimum Qualifying Score

The minimum qualifying score is determined by expanding the number of allowed mismatches in each set of candidate primers and probes until all possible native sequences are represented (e.g., has a qualifying hit).

J. Other

A score is given to based on other parameters, such as relevance to certain patients (e.g., pediatrics, immunocompromised) or certain therapies (e.g., target those strains that respond to treatment) or epidemiology. The prevalence of an organism/strain and the number of times it has been tested for in the community can add value to the selection of the candidate sequences. If a particular strain is more commonly tested then selection of it would be more likely. Strain identification can be used to selection better vaccines.

### Example 2

### Primer/Probe Evaluation

Once the candidate primers and probes have received their scores and have been ranked, they are evaluated using any of a number of methods of the invention, such as BLAST analysis and secondary structure analysis.

A. BLAST Analysis

The candidate primer/probe sets are submitted to BLAST analysis to check for possible overlap with any published sequences that might be missed by the Include/Exclude function. It also provides a useful summary.

B. Secondary Structure

The methods of the present invention include analysis of nucleic acid secondary structure. This includes the structures of the primers and/or probes as well as their intended target variant sequences. The methods and software of the invention predict the optimal temperatures for the annealing but assumes that the target (e.g., RNA or DNA) does not have any significant secondary structure. For example, if the starting material is RNA, the first stage is the creation of a complimentary strand of DNA (cDNA) using a specific primer. This is usually performed at temperatures where the RNA template can have significant secondary structure thereby preventing the annealing of the primer. Similarly, after denaturation of a double stranded DNA target (for example, an amplicon after PCR), the binding of the probe is dependent on there being no major secondary structure in amplicon.

The methods of the invention can either use this information as a criteria for selecting primers and probes or evaluate any secondary structure of a selected sequence, for example, by cutting and pasting candidate primer or probe sequences into a commercial internet link that uses software dedicated to analyzing secondary structure, such as, for example, MFOLD (Zuker et al. (1999) Algorithms and Thermodynamics for RNA Secondary Structure Prediction: A Practical Guide in RNA Biochemistry and Biotechnology, J. Barciszewski and B. F. C. Clark, eds., NATO ASI Series, Kluwer Academic Publishers).

C. Evaluating the Primer and Probe Sequences

The methods and software of the invention may also analyze any nucleic acid sequence to determine its suitability in a nucleic acid amplification-based assay. For example, it can accept a competitor's primer set and determine the following information: (1) How it compares to the primers of the invention (e.g., overall rank, PCR and conservation ranking, etc.); (2) How it aligns to the exclude libraries (e.g., assessing cross-hybridization)—also used to compare primer and probe sets to newly published sequences; and (3) If the sequence has been previously published. This step requires keeping a database of sequences published in scientific journals, posters, and other presentations.

### Example 3

### Multiplexing

The Exclude/Include capability is ideally suited for designing multiplex reactions. The parameters for designing multiple primer and probe sets adhere to a more stringent set of parameters than those used for the initial Exclude/Include function. Each set of primers and probe, together with the resulting amplicon is screened against the other sets that constitute the multiplex reaction. As new targets are accepted their sequences are automatically added to the Exclude category.

The database is designed to interrogate the online databases to determine and acquire, if necessary, any new sequences relevant to the targets. These sequences are evaluated against the optimal primer/probe set. If they represented a new genotype or strain then a multiple sequence alignment may be required.

### Example 4

### Sequences Identified for Detecting HSV

A set of primers and probes useful for detecting and typing HSV variants was generated. The set of primers and probes were then scored according to the methods described herein to identify the optimized primers and probes of Tables 3 and 4. It should be noted that the primers, as they are sequences that anneal to a plurality or all known or unknown HSV variants, can also be used as probes either in the presence or absence of amplification of a sample.

TABLE 3

| Group | | | |
|---|---|---|---|
| no. | Forward Primer | Probe | Reverse Primer |
| 1 | CTGGGCGAGAACAACGA | ACTCCTCGAAGTACACGTAGCCCCCG | GATGTTCAGGTCGATGAAGGT |
| | (SEQ ID NO: 1) | (SEQ ID NO: 2) | (SEQ ID NO: 3) |
| 2 | GCCACGGTGGTGCAGTT | CCTTGAAGACCACCGCGATGCC | TGTACGGGGCGATGTTCT |
| | (SEQ ID NO: 4) | (SEQ ID NO: 5) | (SEQ ID NO: 6) |
| 3 | GCCACGGTGGTGCAGTT | AAGACCACCGCGATGCCCTCC | TGTACGGGGCGATGTTCT |
| | (SEQ ID NO: 4) | (SEQ ID NO: 7) | (SEQ ID NO: 6) |
| 4 | GCCACGGTGGTGCAGTT | ACCGCGATGCCCTCCGTGTAGTTCTG | TGTACGGGGCGATGTTCT |
| | (SEQ ID NO: 4) | (SEQ ID NO: 8) | (SEQ ID NO: 6) |
| 5 | GCCACGGTGGTGCAGTT | TCCTTGAAGACCACCGCGATGCC | TTGTACGGGGCGATGTTC |
| | (SEQ ID NO: 4) | (SEQ ID NO: 9) | (SEQ ID NO: 10) |
| 6 | GCCACGGTGGTGCAGTT | AAGACCACCGCGATGCCCTCCG | TTTGTAGTACATGGTGGCCTTGAA |
| | (SEQ ID NO: 4) | (SEQ ID NO: 11) | (SEQ ID NO: 12) |
| 7 | GCCACGGTGGTGCAGTT | CCTTGAAGACCACCGCGATGCC | TTGTACGGGGCGATGTTCT |
| | (SEQ ID NO: 4) | (SEQ ID NO: 5) | (SEQ ID NO: 13) |
| 8 | GCCACGGTGGTGCAGTT | AAGACCACCGCGATGCCCTCCG | CTTTGTAGTACATGGTGGCCTTGA |
| | (SEQ ID NO: 4) | (SEQ ID NO: 11) | (SEQ ID NO: 14) |
| 9 | GCCACGGTGGTGCAGTT | TCTTCAAGGAGAACATCGCCCCGT | TTTGTAGTACATGGTGGCCTTGAA |
| | (SEQ ID NO: 4) | (SEQ ID NO: 15) | (SEQ ID NO: 12) |
| 10 | GCATCGCGGTGGTCTTC | CAGGTGTGGTTCGGCCACCGCTACT | AAAACGGGGACATGTACACAA |
| | (SEQ ID NO: 16) | (SEQ ID NO: 17) | (SEQ ID NO: 19) |
| 11 | TCAAGGCCACCATGTACTACAAA | CAGGTGTGGTTCGGCCACCGCTAC | GTAAAACGGGGACATGTACACAA |
| | (SEQ ID NO: 20) | (SEQ ID NO: 21) | (SEQ ID NO: 22) |
| 12 | TCAAGGCCACCATGTACTACAAA | AGGTGTGGTTCGGCCACCGCTACTC | GTAAAACGGGGACATGTACACAA |
| | (SEQ ID NO: 20) | (SEQ ID NO: 23) | (SEQ ID NO: 22) |
| 13 | CAAGGCCACCATGTACTACAAAGA | CAGGTGTGGTTCGGCCACCGCTAC | GTAAAACGGGGACATGTACACAAA |
| | (SEQ ID NO: 24) | (SEQ ID NO: 21) | (SEQ ID NO: 68) |
| 14 | GAACATCGCCCCGTACAA | CAGGTGTGGTTCGGCCACCGCTACT | TGTCGATCACCTCCTCGAA |
| | (SEQ ID NO: 25) | (SEQ ID NO: 17) | (SEQ ID NO: 18) |
| 15 | GAACATCGCCCCGTACAA | AGGTGTGGTTCGGCCACCGCTACTC | TGTCGATCACCTCCTCGAA |
| | (SEQ ID NO: 25) | (SEQ ID NO: 23) | (SEQ ID NO: 18) |
| 16 | GAACATCGCCCCGTACAA | TGTGGTTCGGCCACCGCTACTCC | TGTCGATCACCTCCTCGAA |
| | (SEQ ID NO: 25) | (SEQ ID NO: 26) | (SEQ ID NO: 18) |
| 17 | GAACATCGCCCCGTACAA | TGGTTCGGCCACCGCTACTCCCA | TGTCGATCACCTCCTCGAA |
| | (SEQ ID NO: 25) | (SEQ ID NO: 27) | (SEQ ID NO: 18) |
| 18 | GAACATCGCCCCGTACAA | TTCGGCCACCGCTACTCCCAGTTTATG | TGTCGATCACCTCCTCGAA |
| | (SEQ ID NO: 25) | (SEQ ID NO: 28) | (SEQ ID NO: 18) |
| 19 | GAACATCGCCCCGTACAA | CGGCCACCGCTACTCCCAGTTTATGG | TGTCGATCACCTCCTCGAA |
| | (SEQ ID NO: 25) | (SEQ ID NO: 29) | (SEQ ID NO: 18) |
| 20 | CAAGGCCACCATGTACTACAAAGAC | CAGGTGTGGTTCGGCCACCGCTAC | CCCGCGAGGGGTTGTACT |
| | (SEQ ID NO: 65) | (SEQ ID NO: 21) | (SEQ ID NO: 30) |
| 21 | CAAGGCCACCATGTACTACAAAGAC | AGGTGTGGTTCGGCCACCGCTACTC | CCCGCGAGGGGTTGTACT |
| | (SEQ ID NO: 65) | (SEQ ID NO: 23) | (SEQ ID NO: 30) |
| 22 | GCCACGGTGGTGCAGTT | TGGTCTTCAAGGAGAACATCGCCCCGTA | TGTCGATCACCTCCTCGAA |
| | (SEQ ID NO: 4) | (SEQ ID NO: 31) | (SEQ ID NO: 18) |
| 23 | GCCACGGTGGTGCAGTT | TCTTCAAGGAGAACATCGCCCCGTACAA | TGTCGATCACCTCCTCGAA |
| | (SEQ ID NO: 4) | (SEQ ID NO: 32) | (SEQ ID NO: 18) |
| 24 | TCGAGGAGGTGATCGACAA | TGGAGACCACCGCGTTTCACCG | ACGGGGACATGTACACAAAGT |
| | (SEQ ID NO: 33) | (SEQ ID NO: 34) | (SEQ ID NO: 35) |
| 25 | ACTTTGTGTACATGTCCCCGTTTTA | TACGCCGCCGACCGCTTCAA | GGAAGGAGCCGCCGTACTC |
| | (SEQ ID NO: 36) | (SEQ ID NO: 37) | (SEQ ID NO: 38) |
| 26 | ACTTTGTGTACATGTCCCCGTTTTA | CTTCTACGCGCGCGACCTCACCAC | GGAAGGAGCCGCCGTACTC |
| | (SEQ ID NO: 36) | (SEQ ID NO: 67) | (SEQ ID NO: 38) |
| 27 | TTTGTGTACATGTCCCCGTTTTAC | TACGCCGCCGACCGCTTCAA | GAAGGAGCCGCCGTACTC |
| | (SEQ ID NO: 42) | (SEQ ID NO: 37) | (SEQ ID NO: 39) |
| 28 | ACTTTGTGTACATGTCCCCGTTTTAC | TACGCCGCCGACCGCTTCAA | TCCTGCCACTTGGTCATGGT |
| | (SEQ ID NO: 66) | (SEQ ID NO: 37) | (SEQ ID NO: 40) |
| 29 | TCGAGGAGGTGATCGACAA | TGGAGACCACCGCGTTTCACCG | CGCGAGGGGTTGTACTTG |
| | (SEQ ID NO: 33) | (SEQ ID NO: 34) | (SEQ ID NO: 41) |
| 30 | CAAGGCCACCATGTACTACAAA | CAGGTGTGGTTCGGCCACCGCTAC | TGTCGATCACCTCCTCGAA |
| | (SEQ ID NO: 34) | (SEQ ID NO: 21) | (SEQ ID NO: 18) |
| 31 | CAAGGCCACCATGTACTACAAA | AGGTGTGGTTCGGCCACCGCTACTC | TGTCGATCACCTCCTCGAA |
| | (SEQ ID NO: 34) | (SEQ ID NO: 23) | (SEQ ID NO: 18) |
| 32 | CTTCGAGGAGGTGATCGACAAGA | TGGAGACCACCGCGTTTCACCG | CCGCGAGGGGTTGTACTTGA |
| | (SEQ ID NO: 35) | (SEQ ID NO: 34) | (SEQ ID NO: 36) |
| 33 | GAGACCACCGCGTTTCAC | TGGCACACCACCGACCTCAAGTACAACC | AAAACGGGGACATGTACACAA |
| | (SEQ ID NO: 38) | (SEQ ID NO: 39) | (SEQ ID NO: 19) |
| 34 | GAGACCACCGCGTTTCAC | CACACCACCGACCTCAAGTACAACCCCTC | AAAACGGGGACATGTACACAA |
| | (SEQ ID NO: 38) | (SEQ ID NO: 40) | (SEQ ID NO: 19) |
| 35 | GAGACCACCGCGTTTCAC | CCACCGACCTCAAGTACAACCCCTCG | AAAACGGGGACATGTACACAA |
| | (SEQ ID NO: 38) | (SEQ ID NO: 41) | (SEQ ID NO: 19) |
| 36 | GAGACCACCGCGTTTCAC | TGGCACACCACCGACCTCAAGTACAACC | AAACGGGGACATGTACACAAA |
| | (SEQ ID NO: 38) | (SEQ ID NO: 39) | (SEQ ID NO: 42) |
| 37 | CGAGGAGGTGATCGACAAGA | TGGAGACCACCGCGTTTCACCG | CCGCGAGGGGTTGTACT |
| | (SEQ ID NO: 43) | (SEQ ID NO: 34) | (SEQ ID NO: 44) |
| 38 | GGAGACCACCGCGTTTC | TGGCACACCACCGACCTCAAGTACAACC | AGCCGTAAAACGGGGACAT |
| | (SEQ ID NO: 45) | (SEQ ID NO: 39) | (SEQ ID NO: 47) |
| 39 | GGAGACCACCGCGTTTC | CACCACCGACCTCAAGTACAACCCCTCG | AGCCGTAAAACGGGGACAT |
| | (SEQ ID NO: 45) | (SEQ ID NO: 46) | (SEQ ID NO: 47) |
| 40 | CCACGAGACCGACATGGA | TGGCACACCACCGACCTCAAGTACAACC | TAAAACGGGGACATGTACACAA |
| | (SEQ ID NO: 37) | (SEQ ID NO: 39) | (SEQ ID NO: 48) |
| 41 | CTTCGAGGAGGTGATCGACAAGA | TGGAGACCACCGCGTTTCACCG | ACCCGCGAGGGGTTGTACT |
| | (SEQ ID NO: 35) | (SEQ ID NO: 34) | (SEQ ID NO: 49) |
| 42 | TCAAGGCCACCATGTACTACAAAGA | AGGTGTGGTTCGGCCACCGCTACTC | CTTGTCGATCACCTCCTCGAA |
| | (SEQ ID NO: 50) | (SEQ ID NO: 23) | (SEQ ID NO: 52) |
| 43 | CAAGGCCACCATGTACTACAAAGA | CAGGTGTGGTTCGGCCACCGCTAC | TCTTGTCGATCACCTCCTCGAA |
| | (SEQ ID NO: 24) | (SEQ ID NO: 21) | (SEQ ID NO: 53) |
| 44 | CAAGGCCACCATGTACTACAAAGA | AGGTGTGGTTCGGCCACCGCTACTC | TCTTGTCGATCACCTCCTCGAA |
| | (SEQ ID NO: 24) | (SEQ ID NO: 23) | (SEQ ID NO: 53) |
| 45 | CAAGGCCACCATGTACTACAAAGA | TTCGGCCACCGCTACTCCCAGTTTATG | TCTTGTCGATCACCTCCTCGAA |
| | (SEQ ID NO: 24) | (SEQ ID NO: 28) | (SEQ ID NO: 53) |
| 46 | TTCGAGGAGGTGATCGACAAGAT | TGGAGACCACCGCGTTTCACCG | CCGCGAGGGGTTGTACTTGA |
| | (SEQ ID NO: 58) | (SEQ ID NO: 34) | (SEQ ID NO: 36) |
| 47 | CCACGAGACCGACATGGA | CACCACCGACCTCAAGTACAACCCCTCG | GTAAAACGGGGACATGTACACAA |
| | (SEQ ID NO: 37) | (SEQ ID NO: 46) | (SEQ ID NO: 22) |
| 48 | CCACGAGACCGACATGGA | TGGCACACCACCGACCTCAAGTACAACC | ACGGGGACATGTACACAAAGT |
| | (SEQ ID NO: 37) | (SEQ ID NO: 39) | (SEQ ID NO: 35) |
| 49 | AGCGGCCTGCTGGACTAC | AGGTCCAGCGCCGCAACCAG | GGATGACCGTGTCGATGTC |
| | (SEQ ID NO: 54) | (SEQ ID NO: 55) | (SEQ ID NO: 56) |
| 50 | CCACGAGACCGACATGGA | TGGCACACCACCGACCTCAAGTACAACC | AACGGGGACATGTACACAAAGT |
| | (SEQ ID NO: 37) | (SEQ ID NO: 39) | (SEQ ID NO: 57) |
| 51 | TTCGAGGAGGTGATCGACAAGAT | TGGAGACCACCGCGTTTCACCG | ACCCGCGAGGGGTTGTACTT |
| | (SEQ ID NO: 58) | (SEQ ID NO: 34) | (SEQ ID NO: 61) |
| 52 | TGGAGACCACCGCGTTT | CACACCACCGACCTCAAGTACAACCCCTC | CCTCCCGGTAGCCGTAAA |
| | (SEQ ID NO: 59) | (SEQ ID NO: 40) | (SEQ ID NO: 62) |
| 53 | TGGAGACCACCGCGTTT | CACCGACCTCAAGTACAACCCCTCGC | CCTCCCGGTAGCCGTAAA |
| | (SEQ ID NO: 59) | (SEQ ID NO: 60) | (SEQ ID NO: 62) |
| 54 | AGCGGCCTGCTGGACTA | CCGCAACCAGCTGCACGACCT | GGATGACCGTGTCGATGTC |
| | (SEQ ID NO: 64) | (SEQ ID NO: 63) | (SEQ ID NO: 56) |

TABLE 4
Typing Multiplex sets

| No. | run | Forward Primer | Probe | Reverse Primer |
|---|---|---|---|---|
| 1 | 61 | CATTTTACGAGGAGGAGGGGTATAA | AAGCTTCAGCGCGAACGACCAACT | AATCACGGCCCCCAACCT |
| | 62 | AAGACGCCCCTCCCTGTGT | TCAGTCGACCCAAGCGCGGAA | CATCTCGTCGGGGGGAGTAG |
| 2 | 61 | TACGAGGAGGAGGGGTATAACAA | AAGCTTCAGCGCGAACGACCAACT | ATCACGGCCCCCAACCT |
| | | | | (SEQ ID NO: 72) |
| | 62 | TACTCCCCCCGACGAGATG | CCACACAAGCCGCAACGGTCG | GGAGGCGACTGCCGTTT |
| 3 | 61 | GGGGGAGGGGCCATTT | CGGTCATAAGCTTCAGCGCGAACGA | ATCACGGCCCCCAACCT |
| | | | | (SEQ ID NO: 72) |
| | 62 | ACTCCCCCCGACGAGATG | CCACACAAGCCGCAACGGTCG | GGAGGCGACTGCCGTTT |
| 4 | 61 | GGGGGAGGGGCCATTT | TCATAAGCTTCAGCGCGAACGACCAA | ATCACGGCCCCCAACCT |
| | | | | (SEQ ID NO: 72) |
| | 62 | ACTACTCCCCCCGACGAGAT | CCACACAAGCCGCAACGGTCG | GGAGGCGACTGCCGTTT |
| 5 | 61 | GGGAGGGGCCATTTTACG | ATAAGCTTCAGCGCGAACGACCAACTAC | TCACGGCCCCCAACCT |
| | 62 | CCCCCGCAACCACTACTC | CCACACAAGCCGCAACGGTCG | GGAGGCGACTGCCGTTT |
| 6 | 61 | GGGAGGGGCCATTTTACG | CATAAGCTTCAGCGCGAACGACCAACTA | TCACGGCCCCCAACCT |
| | 62 | CTACTCCCCCCGACGAGAT | CCACACAAGCCGCAACGGTCG | GGAGGCGACTGCCGTTT |
| 7 | 61 | CATTTTACGAGGAGGAGGGGTATAA | AAGCTTCAGCGCGAACGACCAACT | AATCACGGCCCCCAACCT |
| | 62 | AAGACGCCCCTCCCTGTGT | TCAGTCGACCCAAGCGCGGAA | TCTCGTCGGGGGGAGTAGTG |
| | | | | (SEQ ID NO: 75) |
| 8 | 61 | GGGAGGGGCCATTTTACG | AAGCTTCAGCGCGAACGACCAACTAC | TCACGGCCCCCAACCT |
| | 62 | TACTCCCCCCGACGAGATG | CCACACAAGCCGCAACGGTCG | GGAGGCGACTGCCGTTT |
| 9 | 61 | GGGGGAGGGGCCATTT | TCATAAGCTTCAGCGCGAACGACCAACTA | ATCACGGCCCCCAACCT |
| | | | | (SEQ ID NO: 72) |
| | 62 | ACTACTCCCCCCGACGAGAT | CCACACAAGCCGCAACGGTCG | GGAGGCGACTGCCGTTT |
| 10 | 61 | GGGGAGGGGCCATTTTAC | AAGCTTCAGCGCGAACGACCAACTA | ATCACGGCCCCCAACCT |
| | | | (SEQ ID NO: 71) | (SEQ ID NO: 72) |
| | 62 | ACTCCCCCCGACGAGAT | CCACACAAGCCGCAACGGTCG | GGAGGCGACTGCCGTTT |
| 11 | 61 | ACGAGGAGGAGGGGTATAACAAA | AAGCTTCAGCGCGAACGACCAAC | ATCACGGCCCCCAACCT |
| | | | | (SEQ ID NO: 72) |
| | 62 | AAGACGCCCCTCCCTGTGT | TCAGTCGACCCAAGCGCGGAAC | CATCTCGTCGGGGGGAGTA |
| | | | (SEQ ID NO: 74) | |
| 12 | 61 | GGGGGAGGGGCCATTT | AAGCTTCAGCGCGAACGACCAACT | TCACGGCCCCCAACCT |
| | 62 | CCCCCGCAACCACTACTC | CCACACAAGCCGCAACGGTCG | GGAGGCGACTGCCGTTT |
| 13 | 61 | GGGAGGGGCCATTTTACG | CATAAGCTTCAGCGCGAACGACCAAC | TCACGGCCCCCAACCT |
| | 62 | ACTACTCCCCCCGACGAGAT | CCACACAAGCCGCAACGGTCG | GGAGGCGACTGCCGTTT |
| 14 | 61 | GGGAGGGGCCATTTTACG | CATAAGCTTCAGCGCGAACGACCAACT | TCACGGCCCCCAACCT |
| | 62 | CCCCCGCAACCACTACTC | CCACACAAGCCGCAACGGTCG | GGAGGCGACTGCCGTTT |
| 15 | 61 | GGGAGGGGCCATTTTACG | CATAAGCTTCAGCGCGAACGACCAACT | TCACGGCCCCCAACCT |
| | 62 | ACTCCCCCCGACGAGAT | CCACACAAGCCGCAACGGTCG | GGAGGCGACTGCCGTTT |
| 16 | 61 | CGAGGAGGAGGGGTATAACAAA | AGCTTCAGCGCGAACGACCAACT | ATCACGGCCCCCAACCT |
| | | (SEQ ID NO: 70) | | (SEQ ID NO: 72) |
| | 62 | AGACGCCCCTCCCTGTGT | CAGTCGACCCAAGCGCGGAA | TCGTCGGGGGGAGTAGTG |
| | | (SEQ ID NO: 73) | | |
| 17 | 61 | CGAGGAGGAGGGGTATAACAAA | AGCTTCAGCGCGAACGACCAACT | ATCACGGCCCCCAACCT |
| | | (SEQ ID NO: 70) | | (SEQ ID NO: 72) |
| | 62 | AGACGCCCCTCCCTGTGT | TCAGTCGACCCAAGCGCGGAA | CATCTCGTCGGGGGGAGTA |
| | | (SEQ ID NO: 73) | | |
| 18 | 61 | ACGAGGAGGAGGGGTATAACAAA | AAGCTTCAGCGCGAACGACCAAC | ATCACGGCCCCCAACCT |
| | | | | (SEQ ID NO: 72) |
| | 62 | AAGACGCCCCTCCCTGTGT | CTCAGTCGACCCAAGCGCGGA | ATCTCGTCGGGGGGAGTAGT |
| 19 | 61 | ACGAGGAGGAGGGGTATAACAAA | AAGCTTCAGCGCGAACGACCAACT | ATCACGGCCCCCAACCT |
| | | | | (SEQ ID NO: 72) |
| | 62 | ACTACTCCCCCCGACGAGAT | CCACACAAGCCGCAACGGTCG | GGAGGCGACTGCCGTTT |
| 20 | 61 | CGAGGAGGAGGGGTATAACAAAG | AAGCTTCAGCGCGAACGACCAACT | ATCACGGCCCCCAACCT |
| | | | | (SEQ ID NO: 72) |
| | 62 | TACTCCCCCCGACGAGATG | CCACACAAGCCGCAACGGTCG | GGAGGCGACTGCCGTTT |
| 21 | 61 | CGAGGAGGAGGGGTATAACAAA | AAGCTTCAGCGCGAACGACCAAC | ATCACGGCCCCCAACCT |
| | | (SEQ ID NO: 70) | | (SEQ ID NO: 72) |
| | 62 | AGACGCCCCTCCCTGTGT | CAGTCGACCCAAGCGCGGAAC | TCTCGTCGGGGGGAGTAGT |
| | | (SEQ ID NO: 73) | | |
| 22 | 61 | ACCTGCGGCTCGTGAAGA | AAACGACTGGACGGAGATTACACAGTTTATCC | CGTCACCCCCTGCTGGTA |
| | 62 | CGTCAGCCCATCCTCCTT | CCGTCCCCAAAGACGTGCGG | CAGCAGGGAAGCATTTACGA |
| | | (SEQ ID NO: 79) | (SEQ ID NO: 80) | (SEQ ID NO: 81) |
| 23 | 61 | AAACGACTGGACGGAGATTACAC | AGTTTATCCTGGAGCACCGAGCCAAG | CGTCACCCCCTGCTGGTA |
| | 62 | CGTCAGCCCATCCTCCTT | CCGTCCCCAAAGACGTGCGG | CAGCAGGGAAGCATTTACGA |
| | | (SEQ ID NO: 79) | (SEQ ID NO: 80) | (SEQ ID NO: 81) |
| 24 | 61 | AAGATAAACGACTGGACGGAGATT | ACACAGTTTATCCTGGAGCACCGAGCC | CGTCACCCCCTGCTGGTA |
| | 62 | CGTCAGCCCATCCTCCTT | CCGTCCCCAAAGACGTGCGG | CAGCAGGGAAGCATTTACGA |
| | | (SEQ ID NO: 79) | (SEQ ID NO: 80) | (SEQ ID NO: 81) |
| 25 | 61 | ATAAACGACTGGACGGAGATTACAC | AGTTTATCCTGGAGCACCGAGCCAAG | CGTCACCCCCTGCTGGTA |
| | 62 | CGTCAGCCCATCCTCCTT | CCGTCCCCAAAGACGTGCGG | CAGCAGGGAAGCATTTACGA |
| | | (SEQ ID NO: 79) | (SEQ ID NO: 80) | (SEQ ID NO: 81) |
| 26 | 61 | GCCCCGCTGGAACTACTATG | ACAGCTTCAGCGCCGTCAGCGA | GTGCCGGCGGTCTCAA |
| | 62 | CGTCAGCCCATCCTCCTT | CCGTCCCCAAAGACGTGCGG | CAGCAGGGAAGCATTTACGA |
| | | (SEQ ID NO: 79) | (SEQ ID NO: 80) | (SEQ ID NO: 81) |
| 27 | 61 | GCCCCGCTGGAACTACTATG | ACAGCTTCAGCGCCGTCAGCGA | GTGTAATCTCCGTCCAGTCGTTTA |
| | 62 | CGTCAGCCCATCCTCCTT | CCGTCCCCAAAGACGTGCGG | CAGCAGGGAAGCATTTACGA |
| | | (SEQ ID NO: 79) | (SEQ ID NO: 80) | (SEQ ID NO: 81) |
| 28 | 61 | GCCCCGCTGGAACTACTATG | ACAGCTTCAGCGCCGTCAGCGA | CCGGCGGTCTCAAACG |
| | 62 | GCCGTCAGCCCATCCT | CCGTCCCCAAAGACGTGCGG | CAGCAGGGAAGCATTTACGA |
| | | | (SEQ ID NO: 80) | (SEQ ID NO: 81) |
| 29 | 61 | CGAAGACGTCCGGAAACAAC | ACAGTTGCCTCCCATCCGAAACCAAG | ATGACCGTGATGGGGATAGC |
| | 62 | CGTCAGCCCATCCTCCTT | CCGTCCCCAAAGACGTGCGG | CAGCAGGGAAGCATTTACGA |
| | | (SEQ ID NO: 79) | (SEQ ID NO: 80) | (SEQ ID NO: 81) |
| 30 | 61 | CCGGAAACAACCCTACAACCT | ACAGTTGCCTCCCATCCGAAACCAAG | ATGACCGTGATGGGGATAGC |
| | | (SEQ ID NO: 82) | | |
| | 62 | CGTCAGCCCATCCTCCTT | CCGTCCCCAAAGACGTGCGG | CAGCAGGGAAGCATTTACGA |
| | | (SEQ ID NO: 79) | (SEQ ID NO: 80) | (SEQ ID NO: 81) |
| 31 | 61 | CGAAGACGTCCGGAAACAA | ACAGTTGCCTCCCATCCGAAACCAAG | ATGACCGTGATGGGGATAGC |
| | 62 | GCCGTCAGCCCATCCT | CCGTCCCCAAAGACGTGCGG | CAGCAGGGAAGCATTTACGA |
| | | | (SEQ ID NO: 80) | (SEQ ID NO: 81) |
| 32 | 61 | GAGGCCCCCCAGATTGTC | ACAGTTGCCTCCCATCCGAAACCAAG | ATGACCGTGATGGGGATAGC |
| | 62 | CGTCAGCCCATCCTCCTT | CCGTCCCCAAAGACGTGCGG | CAGCAGGGAAGCATTTACGA |
| | | (SEQ ID NO: 79) | (SEQ ID NO: 80) | (SEQ ID NO: 81) |
| 33 | 61 | TCCGAAGACGTCCGGAAA | ACAGTTGCCTCCCATCCGAAACCAAG | ATGACCGTGATGGGGATAGC |
| | 62 | CGTCAGCCCATCCTCCTT | CCGTCCCCAAAGACGTGCGG | CAGCAGGGAAGCATTTACGA |
| | | (SEQ ID NO: 79) | (SEQ ID NO: 80) | (SEQ ID NO: 81) |
| 34 | 61 | CCTCCCGATCACGGTTTAC | CCTGCCGCAGCGTGCTCCTA | CCCCGCGGACAATCTG |
| | 62 | CGTCAGCCCATCCTCCTT | CCGTCCCCAAAGACGTGCGG | CAGCAGGGAAGCATTTACGA |
| | | (SEQ ID NO: 79) | (SEQ ID NO: 80) | (SEQ ID NO: 81) |
| 35 | 61 | CCTCCCGATCACGGTTTAC | CCTGCCGCAGCGTGCTCCTA | CCCCGCGGACAATCTG |
| | 62 | GCCGTCAGCCCATCCT | CCGTCCCCAAAGACGTGCGG | CAGCAGGGAAGCATTTACGA |
| | | | (SEQ ID NO: 80) | (SEQ ID NO: 81) |
| 36 | 61 | AGCCCCGCTGGAACTACTATG | CAGCTTCAGCGCCGTCAGCGAG | ATCTTCACGAGCCGCAGGTA |
| | 62 | ACGGCCTCCCCTGCTCTA | ATATCCTCTTTATCATCAGCACCACCATCCACAC | AAGGCGACCAGACAAACGAA |
| 37 | 61 | AGCCCCGCTGGAACTACTATG | CAGCTTCAGCGCCGTCAGCGAG | ATCTTCACGAGCCGCAGGTA |
| | 62 | CTCCCCTGCTCTAGATATCCTCTTT | ATCATCAGCACCACCATCCACACGG | AAGGCGACCAGACAAACGAA |
| 38 | 61 | AGCCCCGCTGGAACTACTATG | CAGCTTCAGCGCCGTCAGCGAG | ATCTTCACGAGCCGCAGGTA |
| | 62 | CTCCCCTGCTCTAGATATCCTCTTTAT | CATCAGCACCACCATCCACACGGC | AAGGCGACCAGACAAACGAA |
| 39 | 61 | AGCCCCGCTGGAACTACTATG | CAGCTTCAGCGCCGTCAGCGAG | ATCTTCACGAGCCGCAGGTA |
| | 62 | TCCCCTGCTCTAGATATCCTCTTTATC | ATCAGCACCACCATCCACACGGC | AAGGCGACCAGACAAACGAA |
| 40 | 61 | AGCCCCGCTGGAACTACTATG | CAGCTTCAGCGCCGTCAGCGAG | ATCTTCACGAGCCGCAGGTA |
| | 62 | CCCTGCTCTAGATATCCTCTTTATCATC | AGCACCACCATCCACACGGCGG | AAGGCGACCAGACAAACGAA |
| 41 | 61 | AGCCCCGCTGGAACTACTATG | CAGCTTCAGCGCCGTCAGCGAG | ATCTTCACGAGCCGCAGGTA |
| | 62 | ACGGCCTCCCCTGCTCTA | ATATCCTCTTTATCATCAGCACCACCATCCACAC | CAAGGCGACCAGACAAACG |
| 42 | 61 | AGCCCCGCTGGAACTACTATG | CAGCTTCAGCGCCGTCAGCGAG | ATCTTCACGAGCCGCAGGTA |
| | 62 | CACATCCCCCTGTTCTGGTT | CCTAACGGCCTCCCCTGCTCTAGATATCCTC | GGATGGTGGTGCTGATGATAAA |
| 43 | 61 | AGCCCCGCTGGAACTACTATG | CAGCTTCAGCGCCGTCAGCGAG | ATCTTCACGAGCCGCAGGTA |
| | 62 | CCCAACACATCCCCCTGTT | CTGGTTCCTAACGGCCTCCCCTGCTCTA | GGATGGTGGTGCTGATGATAAA |
| 44 | 61 | AGCCCCGCTGGAACTACTATG | CAGCTTCAGCGCCGTCAGCGAG | ATCTTCACGAGCCGCAGGTA |
| | 62 | ACACATCCCCCTGTTCTGGTT | CCTAACGGCCTCCCCTGCTCTAGATATCCTCT | TGGATGGTGGTGCTGATGATAA |
| 45 | 61 | AGCCCCGCTGGAACTACTATG | CAGCTTCAGCGCCGTCAGCGAG | ATCTTCACGAGCCGCAGGTA |
| | 62 | GGCGGCGTTCGTTTGTC | TGGTCGCCTTGGCAGCACAACTTT | TCGGGTGCGCGTATCG |
| 46 | 61 | AGCCCCGCTGGAACTACTATG | ACAGCTTCAGCGCCGTCAGCGAG | ATCTTCACGAGCCGCAGGTA |
| | 62 | CCCAACACATCCCCCTGTT | CTGGTTCCTAACGGCCTCCCCTGCTCTA | GGTGGTGCTGATGATAAAGAGGATA |
| 47 | 61 | GCCCCGCTGGAACTACTATG | ACAGCTTCAGCGCCGTCAGCGAG | TCTTCACGAGCCGCAGGTA |
| | 62 | ACGGCCTCCCCTGCTCTA | ATATCCTCTTTATCATCAGCACCACCATCCACAC | TCTTCACGAGCCGCAGGTA |
| 48 | 61 | GCCCCGCTGGAACTACTATG | ACAGCTTCAGCGCCGTCAGCGAG | TCTTCACGAGCCGCAGGTA |
| | 62 | CTCCCCTGCTCTAGATATCCTCTTT | ATCATCAGCACCACCATCCACACGG | AAGGCGACCAGACAAACGAA |
| 49 | 61 | GCCCCGCTGGAACTACTATG | ACAGCTTCAGCGCCGTCAGCGAG | TCTTCACGAGCCGCAGGTA |
| | 62 | CCCTGCTCTAGATATCCTCTTTATCATC | AGCACCACCATCCACACGGCGG | AAGGCGACCAGACAAACGAA |
| 50 | 61 | AGCCCCGCTGGAACTACTATG | ACAGCTTCAGCGCCGTCAGCGAG | TATCTTCACGAGCCGCAGGTA |
| | 62 | ACACATCCCCCTGTTCTGGTT | CCTAACGGCCTCCCCTGCTCTAGATATCCTCTT | CCGCCGTGTGGATGGT |
| | | | TAT | |
| 51 | 61 | ACCTGCGGCTCGTGAAGA | AAACAGACTGGACGGAGATTACACAGTTTATCC | CGTCACCCCCTGCTGGTA |
| | 62 | ACACATCCCCCTGTTCTGGTT | CTAACGGCCTCCCCTGCTCTAGATATCCTCT | GTGGATGGTGGTGCTGATGA |
| 52 | 61 | ACCTGCGGCTCGTGAAGA | AAACGACTGGACGGAGATTACACAGTTTATCC | CGTCACCCCCTGCTGGTA |
| | 62 | ACGGCCTCCCCTGCTCTA | ATATCCTCTTTATCATCAGCACCACCATCCAC | AAGGCGACCAGACAAACGAA |
| 53 | 61 | ACCTGCGGCTCGTGAAGA | AAACGACTGGACGGAGATTACACAGTTTATCC | CGTCACCCCCTGCTGGTA |
| | 62 | CTCCCCTGCTCTAGATATCCTCTTTAT | CATCAGCACCACCATCCACACGG | AAGGCGACCAGACAAACGAA |
| 54 | 61 | ACCTGCGGCTCGTGAAGA | AAACGACTGGACGGAGATTACACAGTTTATCC | CGTCACCCCCTGCTGGTA |
| | 62 | ACGGCCTCCCCTGCTCTA | ATATCCTCTTTATCATCAGCACCACCATCCAC | CAAGGCGACCAGACAAACG |
| 55 | 61 | CCTGCGGCTCGTGAAGAT | AAACGACTGGACGGAGATTACACAGTTTATCC | CGTCACCCCCTGCTGGTA |
| | 62 | ACACATCCCCCTGTTCTGGTT | CTAACGGCCTCCCCTGCTCTAGATATCCTCT | GTGGATGGTGGTGCTGATGA |
| 56 | 61 | CCTGCGGCTCGTGAAGAT | AAACGACTGGACGGAGATTACACAGTTTATCC | CGTCACCCCCTGCTGGTA |
| | 62 | CCCCACACCCCAACACAT | CCCCCTGTTCTGGTTCCTAACGGC | GTGGATGGTGGTGCTGATGA |
| 57 | 61 | CCTGCGGCTCGTGAAGAT | AAACGACTGGACGGAGATTACACAGTTTATCC | CGTCACCCCCTGCTGGTA |
| | 62 | GCCCCACACCCCAACA | CATCCCCCTGTTCTGGTTCCTAACGG | CCGCCGTGTGGATGGT |
| 58 | 61 | AAACGACTGGACGGAGATTACAC | AGTTTATCCTGGAGCACCGAGCCAAG | CGTCACCCCCTGCTGGTA |
| | 62 | ACGGCCTCCCCTGCTCTA | ATATCCTCTTTATCATCAGCACCACCATCCAC | AAGGCGACCAGACAAACGAA |
| 59 | 61 | AAACGACTGGACGGAGATTACAC | AGTTTATCCTGGAGCACCGAGCCAAG | CGTCACCCCCTGCTGGTA |
| | 62 | CTCCCCTGCTCTAGATATCCTCTTTAT | ATCAGCACCACCATCCACACGGC | AAGGCGACCAGACAAACGAA |
| 60 | 61 | GAAGATAAACGACTGGACGGAGAT | ACACAGTTTATCCTGGAGCACCGAGCCAAG | CGTCACCCCCTGCTGGTA |
| | 62 | ACGGCCTCCCCTGCTCTA | ATATCCTCTTTATCATCAGCACCACCATCCACA | AAGGCGACCAGACAAACGAA |
| 61 | 61 | GAAGATAAACGACTGGACGGAGAT | ACACAGTTTATCCTGGAGCACCGAGCCAAG | CGTCACCCCCTGCTGGTA |
| | 62 | ACACATCCCCCTGTTCTGGTT | CCTAACGGCCTCCCCTGCTCTAGATATCCTCTT | TGTGGATGGTGGTGCTGATG |
| | | | TAT | |
| 62 | 61 | GAAGATAAACGACTGGACGGAGAT | TACACAGTTTATCCTGGAGCACCGAGCCAA | CGTCACCCCCTGCTGGTA |
| | 62 | CACATCCCCCTGTTCTGGTT | CCTAACGGCCTCCCCTGCTCTAGATATCCTCTT | CCGCCGTGTGGATGGT |
| | | | TAT | |
| 63 | 61 | AAGATAAACGACTGGACGGAGATT | ACACAGTTTATCCTGGAGCACCGAGCCAAG | CGTCACCCCCTGCTGGTA |
| | 62 | ACGGCCTCCCCTGCTCTA | ATATCCTCTTTATCATCAGCACCACCATCCACAC | AAGGCGACCAGACAAACGAA |
| 64 | 61 | TAAACGACTGGACGGAGATTACAC | AGTTTATCCTGGAGCACCGAGCCAAG | CGTCACCCCCTGCTGGTA |
| | 62 | ACGGCCTCCCCTGCTCTA | ATATCCTCTTTATCATCAGCACCACCATCCAC | AAGGCGACCAGACAAACGAA |
| 65 | 61 | TAAACGACTGGACGGAGATTACAC | AGTTTATCCTGGAGCACCGAGCCAAG | CGTCACCCCCTGCTGGTA |
| | 62 | TCCCCTGCTCTAGATATCCTCTTTATC | ATCAGCACCACCATCCACACGGC | AAGGCGACCAGACAAACGAA |
| 66 | 61 | GATAAACGACTGGACGGAGATTACA | CAGTTTATCCTGGAGCACCGAGCCAAG | CGTCACCCCCTGCTGGTA |
| | 62 | ACGGCCTCCCCTGCTCTA | ATATCCTCTTTATCATCAGCACCACCATCCACAC | AAGGCGACCAGACAAACGAA |
| 67 | 61 | GATAAACGACTGGACGGAGATTACA | CAGTTTATCCTGGAGCACCGAGCCAAG | CGTCACCCCCTGCTGGTA |
| | 62 | CCCCACACCCCAACACAT | CCCCCTGTTCTGGTTCCTAACGGCCT | CCGCCGTGTGGATGGT |
| 68 | 61 | ATAAACGACTGGACGGAGATTACAC | AGTTTATCCTGGAGCACCGAGCCAAG | CGTCACCCCCTGCTGGTA |
| | 62 | ACGGCCTCCCCTGCTCTA | ATATCCTCTTTATCATCAGCACCACCATCCAC | AAGGCGACCAGACAAACGAA |
| 69 | 61 | AAGATAAACGACTGGACGGAGATTA | CACAGTTTATCCTGGAGCACCGAGCCAAG | CGTCACCCCCTGCTGGTA |
| | 62 | ACGGCCTCCCCTGCTCTA | ATATCCTCTTTATCATCAGCACCACCATCCACAC | AAGGCGACCAGACAAACGAA |
| 70 | 61 | GAAGATAAACGACTGGACGGAGATT | ACACAGTTTATCCTGGAGCACCGAGCCAAG | CGTCACCCCCTGCTGGTA |
| | 62 | ACGGCCTCCCCTGCTCTA | ATATCCTCTTTATCATCAGCACCACCATCCACAC | AAGGCGACCAGACAAACGAA |
| 71 | 61 | AAGATAAACGACTGGACGGAGATTAC | CAGTTTATCCTGGAGCACCGAGCCAAG | CGTCACCCCCTGCTGGTA |
| | 62 | ACGGCCTCCCCTGCTCTA | ATATCCTCTTTATCATCAGCACCACCATCCACAC | AAGGCGACCAGACAAACGAA |
| 72 | 61 | AAGATAAACGACTGGACGGAGATTAC | ACAGTTTATCCTGGAGCACCGAGCCAAG | CGTCACCCCCTGCTGGTA |
| | 62 | ACGGCCTCCCCTGCTCTA | ATATCCTCTTTATCATCAGCACCACCATCCACAC | AAGGCGACCAGACAAACGAA |
| 73 | 61 | GAAGATAAACGACTGGACGGAGATTA | CACAGTTTATCCTGGAGCACCGAGCCAAG | CGTCACCCCCTGCTGGTA |
| | 62 | ACGGCCTCCCCTGCTCTA | ATATCCTCTTTATCATCAGCACCACCATCCACAC | AAGGCGACCAGACAAACGAA |
| 74 | 61 | GCCCCGCTGGAACTACTATG | ACAGCTTCAGCGCCGTCAGCGAG | TCCGTCCAGTCGTTTATCTTCA |
| | 62 | ACGGCCTCCCCTGCTCTA | ATATCCTCTTTATCATCAGCACCACCATCCACAC | GCCAAGGCGACCAGACAA |
| 75 | 61 | GCCCCGCTGGAACTACTATG | ACAGCTTCAGCGCCGTCAGCGAG | TCCGTCCAGTCGTTTATCTTCA |
| | 62 | CTCCCCTGCTCTAGATATCCTCTTT | ATCATCAGCACCACCATCCACACGG | GCCAAGGCGACCAGACAA |
| 76 | 61 | GCCCCGCTGGAACTACTATG | ACAGCTTCAGCGCCGTCAGCGAG | TCCGTCCAGTCGTTTATCTTCA |
| | 62 | ACACATCCCCCTGTTCTGGTT | CCTAACGGCCTCCCCTGCTCTAGATATCCTCT | CCGCCGTGTGGATGGT |
| | | | TTAT | |
| 77 | 61 | AGCCCCGCTGGAACTACTATG | ACAGCTTCAGCGCCGTCAGCGAG | TCCGTCCAGTCGTTTATCTTCA |
| | 62 | ACGGCCTCCCCTGCTCTA | ATATCCTCTTTATCATCAGCACCACCATCCACAC | GCCAAGGCGACCAGACAA |
| 78 | 61 | AGCCCCGCTGGAACTACTATG | CAGCTTCAGCGCCGTCAGCGAG | TCCGTCCAGTCGTTTATCTTCAC |
| | 62 | CCTGCTCTAGATATCCTCTTTATCATCAG | ACCACCATCCACACGGCGGC | GCCAAGGCGACCAGACAA |
| 79 | 61 | GCGCGTGTACCACATCCA | CCCCAGCCTCCCGATCACGGTTTAC | CCGACGGTGCGTTTAGGA |
| | 62 | ACTACTCCCCCCGACGAGAT | CCACACAAGCCGCAACGGTCG | GGAGGCGACTGCCGTTT |
| 80 | 61 | GCCTCCCGATCACGGTTTA | CGCAGCGTGCTCCTAAACGCACC | CCCCGCGGACAATCTG |
| | 62 | ACTACTCCCCCCGACGAGAT | CCACACAAGCCGCAACGGTCG | GGAGGCGACTGCCGTTT |
| 81 | 61 | GCCTCCCGATCACGGTTTA | CCTGCCGCAGCGTGCTCCTAA | CCCCGCGGACAATCTG |
| | 62 | CCCCCGCAACCACTACTC | CCACACAAGCCGCAACGGTCG | GGAGGCGACTGCCGTTT |
| 82 | 61 | CCTCCCGATCACGGTTTAC | CGCAGCGTGCTCCTAAACGCACC | CCCCGCGGACAATCTG |
| | 62 | ACTACTCCCCCCGACGAGAT | CCACACAAGCCGCAACGGTCG | GGAGGCGACTGCCGTTT |

The following combinations are used for testing multiplexed sets for HSV typing.

| Non-typing set A | |
|---|---|
| Target = glycoprotein B gene | |
| Forward Primer | GCCACGGTGGTGCAGTT |
| | (SEQ ID NO: 4) |
| Probe | CCGCGATGCCCTCCGTGTAGTTC |
| | (SEQ ID NO: 69) |
| Reverse Primer | TTGTACGGGGCGATGTTC |
| | (SEQ ID NO: 10) |
| Non-typing set B | |
| Target = glycoprotein B gene | |
| Forward Primer | TCAAGGCCACCATGTACTACAAA |
| | (SEQ ID NO: 20) |
| Probe | CAGGTGTGGTTCGGCCACCGCTAC |
| | (SEQ ID NO: 21) |
| Reverse Primer | CTTGTCGATCACCTCCTCGAA |
| | (SEQ ID NO: 52) |
| Typing set A | |
| HSV-1 target = glycoprotein D | |
| Forward Primer | CGAGGAGGAGGGGTATAACAAA |
| | (SEQ ID NO: 70) |
| Probe | AAGCTTCAGCGCGAACGACCAACTA |
| | (SEQ ID NO: 71) |
| Reverse Primer | ATCACGGCCCCCAACCT |
| | (SEQ ID NO: 72) |
| HSV-2 target = glycoprotein G gene | |
| Forward Primer | AGACGCCCCTCCCTGTGT |
| | (SEQ ID NO: 73) |
| Probe | TCAGTCGACCCAAGCGCGGAAC |
| | (SEQ ID NO: 74) |
| Reverse Primer | CTCGTCGGGGGGAGTAGTG |
| | (SEQ ID NO: 75) |
| Typing set B | |
| HSV-1 target = glycoprotein D gene | |
| Forward Primer | TCCGAAGACGTCCGGAAA |
| | (SEQ ID NO: 76) |
| Probe | CCTCCCATCCGAAACCAAGCGATG |
| | (SEQ ID NO: 77) |
| Reverse Primer | CGTGATGGGGATAGCACAGTT |
| | (SEQ ID NO: 78) |
| HSV-2 target = glycoprotein G gene | |
| Forward Primer | CGTCAGCCCATCCTCCTT |
| | (SEQ ID NO: 79) |
| Probe | CCGTCCCCAAAGACGTGCGG |
| | (SEQ ID NO: 80) |
| Reverse Primer | CAGCAGGGAAGCATTTACGA |
| | (SEQ ID NO: 81) |
| Typing set C | |
| HSV-1 target = glycoprotein D gene | |
| Forward Primer | CCGGAAACAACCCTACAACCT |
| | (SEQ ID NO: 82) |
| Probe | CCTCCCATCCGAAACCAAGCGATG |
| | (SEQ ID NO: 77) |
| Reverse Primer | CGTGATGGGGATAGCACAGTT |
| | (SEQ ID NO: 78) |
| HSV-2 target = glycoprotein G gene | |
| Forward Primer | CGTCAGCCCATCCTCCTT |
| | (SEQ ID NO: 79) |
| Probe | CCGTCCCCAAAGACGTGCGG |
| | (SEQ ID NO: 80) |
| Reverse Primer | CAGCAGGGAAGCATTTACGA |
| | (SEQ ID NO: 81) |
| Typing set D | |
| HSV-1 target = glycoprotein G gene | |
| Forward Primer | GTGCCGTTGTTCCCATTATC |
| | (SEQ ID NO: 83) |
| Probe | CCATTCCTTTTGGTTCTTGTCGGTGTATCG |
| | (SEQ ID NO: 84) |
| Reverse Primer | GGGTGGTGGAGGAGACGTT |
| | (SEQ ID NO: 85) |
| HSV-2 target = glycoprotein G gene | |
| Forward Primer | CGTCAGCCCATCCTCCTT |
| | (SEQ ID NO: 79) |
| Probe | CCGTCCCCAAAGACGTGCGG |
| | (SEQ ID NO: 80) |
| Reverse Primer | CAGCAGGGAAGCATTTACGA |
| | (SEQ ID NO: 81) |
| Typing set E | |
| HSV-1 target = glycoprotein G gene | |
| Forward Primer | GTGCCGTTGTTCCCATTATC |
| | (SEQ ID NO: 83) |
| Probe | CCATTCCTTTTGGTTCTTGTCGGTGTATCG |
| | (SEQ ID NO: 84) |
| Reverse Primer | GGGTGGTGGAGGAGACGTT |
| | (SEQ ID NO: 85) |
| HSV-2 target = glycoprotein G gene | |
| Forward Primer | CGTCAGCCCATCCTCCTT |
| | (SEQ ID NO: 79) |
| Probe | CGACCCGGTACGCTCTCGTAAATGCTTC |
| | (SEQ ID NO: 86) |
| Reverse Primer | CGCCGAGTTCGATCTGGTA |
| | (SEQ ID NO: 87) |
| Typing set F | |
| HSV-1 target = glycoprotein D gene | |
| Forward Primer | CCGGAAACAACCCTACAACCT |
| | (SEQ ID NO: 82) |
| Probe | CCTCCCATCCGAAACCAAGCGATG |
| | (SEQ ID NO: 77) |
| Reverse Primer | CGTGATGGGGATAGCACAGTT |
| | (SEQ ID NO: 78) |
| HSV-2 target = glycoprotein G gene | |
| Forward Primer | AGACGCCCCTCCCTGTGT |
| | (SEQ ID NO: 73) |
| Probe | TCAGTCGACCCAAGCGCGGAAC |
| | (SEQ ID NO: 74) |
| Reverse Primer | CTCGTCGGGGGGAGTAGTG |
| | (SEQ ID NO: 75) |

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the following claims.

What is claimed is:

1. An isolated polynucleotide, comprising a nucleotide sequence that comprises any one of SEQ ID NOs: 1-87.

2. An isolated polynucleotide, comprising any of the nucleotide sequences depicted in Table 3 or any of the nucleotide sequences depicted in Table 4.

3. A primer pair for amplifying herpes simplex virus DNA, comprising a forward and reverse primer selected from the group consisting of the sequences listed in groups 1-54 of Table 3.

4. A primer pair for amplifying herpes simplex virus DNA, comprising the forward and reverse primer pairs depicted in Table 4.

5. A primer pair for amplifying herpes simplex virus DNA selected from the group consisting of (1) SEQ ID NOs: 4 and 10; (2) SEQ ID NOs: 20 and 52; (3) SEQ ID NOs: 70 and 72; (4) SEQ ID NOs: 73 and 75; (5) SEQ ID NOs: 76 and 78; (6) SEQ ID NOs: 79 and 81; (7) SEQ ID NOs: 82 and 78; (8) SEQ ID NOs: 79 and 81; (9) SEQ ID NOs: 83 and 85; and (10) SEQ ID NOs: 79 and 87.

6. A polynucleotide probe that binds to a PCR product created by the primer pair of claim 5, wherein (1) the probe comprising the sequence of SEQ ID NO: 69 hybridizes to the PCR product amplified by SEQ ID NOs: 4 and 10; (2) the probe comprising the sequence of SEQ ID NO: 21 hybridizes to the PCR product amplified by SEQ ID NOs: 20 and 52; (3) the probe comprising the sequence of SEQ ID NO: 71 hybridizes to the PCR product amplified by SEQ ID NOs: 70 and 72; (4) the probe comprising the sequence of SEQ ID NO: 74 hybridizes to the PCR product amplified by SEQ ID NOs: 73 and 75; (5) the probe comprising the sequence of SEQ ID NO: 77 hybridizes to the PCR product amplified by (i) SEQ ID NOs: 76 and 78, and (ii) SEQ ID NOs: 82 and 78; (6) the probe comprising the sequence of SEQ ID NO: 80 hybridizes to the PCR product amplified by SEQ ID NOs: 79 and 81; (7) the probe comprising the sequence of SEQ ID NO: 84 hybridizes to the PCR product amplified by SEQ ID NOs: 83 and 85; (8) the probe comprising the sequence of SEQ ID NO: 84 hybridizes to the PCR product amplified by SEQ ID NOs: 83 and 85; and (9) the probe comprising the sequence of SEQ ID NO: 86 hybridizes to the PCR product amplified by SEQ ID NOs: 79 and 87.

7. The polynucleotide probe of claim 6, wherein the probe is labeled.

8. The polynucleotide probe of claim 7, wherein the probe comprises a fluorescent label, a chemiluminescent label, a radioactive label, biotin, or gold.

9. A method for detecting an HSV virus in a sample, comprising (1) adding together at least once group of forward and reverse primers depicted in Tables 3 or 4 to a sample, (2) conducting a polymerase chain reaction on the sample, and (3) detecting the generation of a PCR product, wherein the generation of an amplified PCR product indicates the presence of an HSV variant in the sample.

10. The method of claim 9, wherein the forward and reverse primers comprise at least one sequence from the group consisting of: (1) SEQ ID NOs: 4 and 10; (2) SEQ ID NOs: 20 and 52; (3) SEQ ID NOs: 70 and 72; (4) SEQ ID NOs: 73 and 75; (5) SEQ ID NOs: 76 and 78; (6) SEQ ID NOs: 79 and 81; (7) SEQ ID NOs: 82 and 78; (8) SEQ ID NOs: 79 and 81; (9) SEQ ID NOs: 83 and 85; and (10) SEQ ID NOs: 79 and 87, respectively.

11. The method of claim 10, further comprising the steps of (1) adding a labeled probe to the sample, wherein the probe comprises the sequence that corresponds to the forward and reverse primer pair group depicted in Tables 3 or 4, and (2) detecting the binding of the probe to an amplified PCR product after exposing the PCR product and probe(s) to conditions that promote hybridization.

12. The method of claim 10, wherein the sequence of the probe or probes is selected from the group consisting of SEQ ID NOs: 21, 69, 71, 74, 77, 80, 84, and 86.

13. The method of claim 9, wherein the probe is fluorescently labeled and the step of detecting the binding of the probe to the amplified PCR product entails measuring the fluorescence of the sample.

14. The method of claim 9, wherein the sample is blood, serum, plasma, sputum, urine, stool, skin, cerebrospinal fluid, saliva, gastric secretions, tears, oropharyngeal swabs, nasopharyngeal swabs, throat swabs, nasal aspirates, nasal wash, and fluids collected from the ear, eye, mouth, respiratory airways, spinal tissue or fluid, cerebral fluid, trigeminal ganglion sample or a sacral ganglion sample.
